(19) United States
(12) Patent Application Publication
(10) Pub. No.: US 2010/0056535 A1
(43) Pub. Date: Mar. 4, 2010

(54) Inhibitors of HIV-1 reverse transcriptase

(75) Inventors: Nidhi Arora, Cupertino, CA (US); Roland Joseph Billedeau, Santa Clara, CA (US); Joshua Kennedy-Smith, San Francisco, CA (US); Weiling Liang, Palo Alto, CA (US); Ralf Roetz, Mountain View, CA (US); Zachary Kevin Sweeney, Redwood City, CA (US)

Correspondence Address: Grant D. Green;Patent Law Department, M/S A2-250, Roche Palo Alto LLC, 3431 Hillview Avenue, Palo Alto, CA, 94304, US

(73) Assignee: Roche Palo Alto LLC

(21) Appl. No.: 12/584,436

(22) Filed: Sep. 4, 2009

Related U.S. Application Data
(60) Provisional application No. 61/094,109, filed on Sep. 4, 2008.

(51) Int. Cl. A61K 31/501; C07D 217/06; C07D 403/06; A61K 31/4709; A61K 31/4439; A61P 31/18

(52) U.S. Cl. 514/252.03; 546/146; 546/271.7; 544/238; 514/307; 514/338

(57) ABSTRACT

The present invention provides compounds for treating or preventing an HIV infection, or treating AIDS or ARC comprising administering a compound according to Formulae I and II

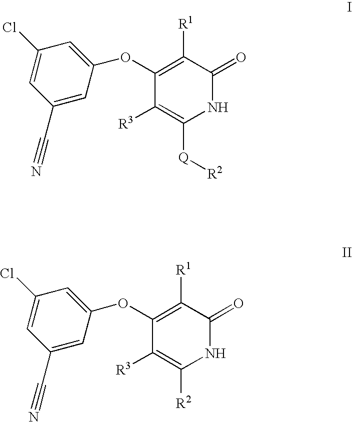

wherein Q, R¹, R², and R³ are defined as described herein.

### CROSS-REFERENCE TO RELATED APPLICATIONS

This application is entitled to the benefit of U.S. provisional patent application Ser. No. 61/094,109 filed on Sep. 4, 2008, the disclosure of which is incorporated herein by reference.

### FIELD OF THE INVENTION

The invention relates to the field of antiviral therapy and, in particular, to non-nucleoside compounds that inhibit HIV reverse transcriptase and are useful for treating Human Immunodeficiency Virus (HIV) mediated diseases. The invention provides novel pyridone compounds according to formula I, for treatment or prophylaxis of HIV mediated diseases, AIDS or ARC, employing said compounds in monotherapy or in combination therapy.

### BACKGROUND OF THE INVENTION

The human immunodeficiency virus HIV is the causative agent of acquired immunodeficiency syndrome (AIDS), a disease characterized by the destruction of the immune system, particularly of the CD4⁺ T-cell, with attendant susceptibility to opportunistic infections. HIV infection is also associated with a precursor AIDS-related complex (ARC), a syndrome characterized by symptoms such as persistent generalized lymphadenopathy, fever and weight loss.

In common with other retroviruses, the HIV genome encodes protein precursors known as gag and gag-pol which are processed by the viral protease to afford the protease, reverse transcriptase (RT), endonuclease/integrase and mature structural proteins of the virus core. Interruption of this processing prevents the production of normally infectious virus. Considerable efforts have been directed towards the control of HIV by inhibition of virally encoded enzymes.

Inhibitors of HIV reverse transcriptase are critical components of commonly used combination antiretroviral therapy (cART). (C. Flexner: HIV drug development: the next 25 years. Nat. Rev. Drug Discov., 2007, 6, 959-966. K. Struble et al., Antiretroviral therapies for treatment experienced patients: current status and research challenges. AIDS, 2005, 19, 747-756.). Two general classes of RTI inhibitors have been identified: nucleoside reverse transcriptase inhibitors (NRTI) and non-nucleoside reverse transcriptase inhibitors.

NRTIs typically are 2′,3′-dideoxynucleoside (ddN) analogs which must be phosphorylated prior to interacting with viral RT. The corresponding triphosphates function as competitive inhibitors or alternative substrates for viral RT. After incorporation into nucleic acids the nucleoside analogs terminate the chain elongation process. HIV reverse transcriptase has DNA editing capabilities which enable resistant strains to overcome the blockade by cleaving the nucleoside analog and continuing the elongation. Currently clinically used NRTls include zidovudine (AZT), didanosine (ddI), zalcitabine (ddC), stavudine (d4T), lamivudine (3TC) and tenofovir (PMPA).

NNRTIs were first discovered in 1989. NNRTI are allosteric inhibitors which bind reversibly at a nonsubstrate-binding site on the HIV reverse transcriptase thereby altering the shape of the active site or blocking polymerase activity (Z. Sweeney and K Klumpp, Improving non-nucleoside reverse transcriptase inhibitors for first-line treatment of HIV infection: The development pipeline and recent clinical data. Curr. Opinion Drug Discov. Development, 2008, 11, 458. Z. Zhang et al. Clinical utility of current NNRTIs and perspectives of new agents in this class under development. Antivir. Chem. Chemother., 2004, 15, 121. N. Sluis-Cremer et al., Mechanisms of inhibition of HIV replication by non-nucleoside reverse transcriptase inhibitors. Virus Res., 2008, 134, 147-156.). Although over thirty structural classes of NNRTIs have been identified in the laboratory, only three compounds have been approved for HIV therapy: efavirenz, nevirapine and delavirdine.

Initially viewed as a promising class of compounds, in vitro and in vivo studies quickly revealed the NNRTIs presented a low barrier to the emergence of drug resistant HIV strains and class-specific toxicity. Drug resistance frequently develops with only a single point mutation in the RT. While combination therapy with NRTIs, PIs and NNRTIs has, in many cases, dramatically lowered viral loads and slowed disease progression, significant therapeutic problems remain. (R. M. Gulick, Eur. Soc. Clin. Microbiol. and Inf. Dis. 2003 9(3):186-193) The cocktails are not effective in all patients, potentially severe adverse reactions often occur and the rapidly reproducing HIV virus has proven adroit at creating mutant drug-resistant variants of wild type protease and reverse transcriptase. There remains a need for safer drugs with activity against wild type and commonly occurring resistant strains of HIV.

### SUMMARY OF THE INVENTION

The present invention provides compounds for treating or preventing an HIV infection, or treating AIDS or ARC comprising administering a compound according to Formulae I and II

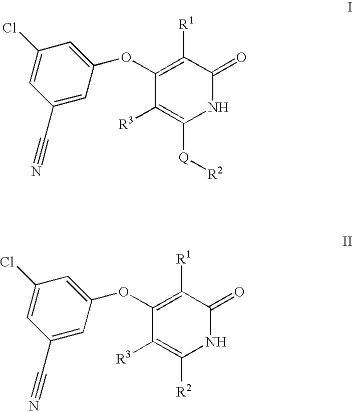

wherein Q, R¹, R², and R³ are defined as described herein.

The application provides a compound of Formula I

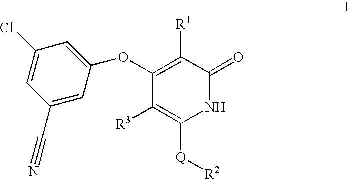

wherein:
R¹ is halogen, lower alkyl, lower alkenyl, or amino;

### Q is Q¹ or Q²;

Q¹ is lower alkylene;

Q² is Q¹-Q³;
Q³ is —(═O)—;
   R² is phenyl, heteroaryl, or heterocycloalkyl, optionally substituted with one or more R^{2′};

R^{2′} is lower alkyl or halogen; and

R³ is H, halogen, or lower alkyl.

In one embodiment of Formula I, R¹ is halogen.

In one embodiment of Formula I, Q is ethylene.

In one embodiment of Formula I, R¹ is halogen and Q is ethylene.

In one embodiment of Formula I, R² is phenyl.

In one embodiment of Formula I, R² is phenyl and R¹ is halogen.

In one embodiment of Formula I, R² is phenyl, Q is ethylene, and R¹ is halogen.

In one embodiment of Formula I, R² is pyridyl.

In one embodiment of Formula I, R² is pyridyl and R¹ is halogen.

In one embodiment of Formula I, R² is pyridyl, Q is ethylene, and R¹ is halogen.

In one embodiment of Formula I, R³ is H.

In one embodiment of Formula I, R¹ is halogen, and R³ is H.

In one embodiment of Formula I, R² is phenyl, R¹ is halogen, and R³ is H.

In one embodiment of Formula I, R² is phenyl, Q is ethylene, R¹ is halogen, and R³ is H.

In one embodiment of Formula I, R² is pyridyl, R¹ is halogen, and R³ is H.

In one embodiment of Formula I, R² is pyridyl, Q is ethylene, R¹ is halogen, and R³ is H.

In one embodiment of Formula I, R³ is F.

In one embodiment of Formula I, R¹ is halogen, and R³ is F.

In one embodiment of Formula I, R² is phenyl, R¹ is halogen, and R³ is F.

In one embodiment of Formula I, R² is phenyl, Q is ethylene, R¹ is halogen, and R³ is F.

In one embodiment of Formula I, R² is pyridyl, R¹ is halogen, and R³ is F.

In one embodiment of Formula I, R² is pyridyl, Q is ethylene, R¹ is halogen, and R³ is F.

The application also provides the compound of Formula I selected from the group consisting of:
3-Chloro-5-{6-[2-(3,4-dihydro-1H-isoquinolin-2-yl)-2-oxo-ethyl]-3-dimethylamino-2-oxo-1,2-dihydro-pyridin-4-yloxy}-benzonitrile;
3-[6-(2-Benzooxazol-2-yl-ethyl)-3-dimethylamino-5-methyl-2-oxo-1,2-dihydro-pyridin-4-yloxy]-5-chloro-benzonitrile;
3-(3-Bromo-2-oxo-6-phenethyl-1,2-dihydro-pyridin-4-yloxy)-5-chloro-benzonitrile;
3-Chloro-5-(3-chloro-2-oxo-6-phenethyl-1,2-dihydro-pyridin-4-yloxy)-benzonitrile;
3-{3-Bromo-6-[2-(3,4-dihydro-1H-isoquinolin-2-yl)-2-oxo-ethyl]-5-fluoro-2-oxo-1,2-dihydro-pyridin-4-yloxy}-5-chloro-benzonitrile;
3-(3-Bromo-5-fluoro-2-oxo-6-phenethyl-1,2-dihydro-pyridin-4-yloxy)-5-chloro-benzonitrile;
3-Chloro-5-(3-chloro-5-fluoro-2-oxo-6-phenethyl-1,2-dihydro-pyridin-4-yloxy)-benzonitrile;
3-Chloro-5-{3-chloro-6-[2-(2-methyl-pyridin-4-yl)-ethyl]-2-oxo-1,2-dihydro-pyridin-4-yloxy}-benzonitrile;
3-Chloro-5-{6-[2-(3-chloro-phenyl)-ethyl]-5-fluoro-3-iodo-2-oxo-1,2-dihydro-pyridin-4-yloxy}-benzonitrile;
3-Chloro-5-[3-chloro-2-oxo-6-(2-m-tolyl-ethyl)-1,2-dihydro-pyridin-4-yloxy]-benzonitrile;
3-Chloro-5-[3-chloro-2-oxo-6-(2-pyridin-4-yl-ethyl)-1,2-dihydro-pyridin-4-yloxy]-benzonitrile;
3-{3-Bromo-6-[2-(3-chloro-phenyl)-ethyl]-5-fluoro-2-oxo-1,2-dihydro-pyridin-4-yloxy}-5-chloro-benzonitrile;
3-Chloro-5-[3-chloro-2-oxo-6-(2-pyridin-3-yl-ethyl)-1,2-dihydro-pyridin-4-yloxy]-benzonitrile;
3-Chloro-5-[3-chloro-2-oxo-6-(2-pyridin-2-yl-ethyl)-1,2-dihydro-pyridin-4-yloxy]-benzonitrile;
3-Chloro-5-{3-chloro-6-[2-(3-chloro-phenyl)-ethyl]-2-oxo-1,2-dihydro-pyridin-4-yloxy}-benzonitrile;
3-Chloro-5-{3-chloro-6-[2-(3,4-dihydro-1H-isoquinolin-2-yl)-2-oxo-ethyl]-2-oxo-1,2-dihydro-pyridin-4-yloxy}-benzonitrile;
3-{3-Bromo-5-fluoro-6-[2-(3-fluoro-phenyl)-ethyl]-2-oxo-1,2-dihydro-pyridin-4-yloxy}-5-chloro-benzonitrile;
3-Chloro-5-{3-chloro-5-fluoro-6-[2-(3-fluoro-phenyl)-ethyl]-2-oxo-1,2-dihydro-pyridin-4-yloxy}-benzonitrile;
3-[6-(2-Benzooxazol-2-yl-ethyl)-3-chloro-2-oxo-1,2-dihydro-pyridin-4-yloxy]-5-chloro-benzonitrile;
3-[3-Bromo-5-fluoro-2-oxo-6-(2-pyridin-4-yl-ethyl)-1,2-dihydro-pyridin-4-yloxy]-5-chloro-benzonitrile;
3-Chloro-5-[3-chloro-5-fluoro-2-oxo-6-(2-pyridin-4-yl-ethyl)-1,2-dihydro-pyridin-4-yloxy]-benzonitrile; and
3-{3-Bromo-6-[2-(3-chloro-phenyl)-ethyl]-5-fluoro-2-oxo-1,2-dihydro-pyridin-4-yloxy}-5-chloro-benzonitrile.

The application further provides a compound of Formula II

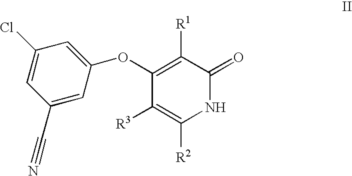

wherein:
R¹ is halogen, lower alkyl, lower alkenyl, or amino;
R¹ is H or lower alkyl;

### R³ is —R⁴ or —R⁵—R⁶;

R⁴ is lower alkyl;

R⁵ is —(CH₂)ₘ—, —(CH₂)ₘO— or —(CH₂)ₘS—;
m is 1, 2, or 3;

R⁶ is phenyl, phenyl lower alkylenyl, heteroaryl, or heteroaryl lower alkylenyl, optionally substituted with one or more R^{6′}; and
R^{6′} is lower alkyl, halogen or lower alkoxy.

In one embodiment of Formula II, R¹ is halogen.

In one embodiment of Formula II, R³ is —R⁵—R⁶, R⁵ is —(CH₂)ₘO—, and m is 2.

In one embodiment of Formula II, R¹ is halogen, R³ is —R⁵—R⁶, R⁵ is —(CH₂)ₘO—, and m is 2.

In one embodiment of Formula II, R⁶ is phenyl, R³ is —R⁵—R⁶, R¹ is —(CH₂)ₘO— and m is 2.

In one embodiment of Formula II, R⁶ is pyridyl, R³ is —R⁵—R⁶, R⁵ is —(CH₂)ₘO— and m is 2.

In one embodiment of Formula II, R⁶ is phenyl, R¹ is halogen, R³ is —R⁵—R⁶, R⁵ is —(CH₂)ₘO—, and m is 2.

In one embodiment of Formula II, R¹ is halogen and R² is lower alkyl.

In one embodiment of Formula II, R³ is —R⁵—R⁶, R⁵ is —(CH₂)ₘO—, m is 2, and R² is lower alkyl.

In one embodiment of Formula II, R¹ is halogen, R³ is —R⁵—R⁶, R⁵ is —(CH₂)ₘO—, m is 2, and R² is lower alkyl.

In one embodiment of Formula II, R⁶ is phenyl, R² is lower alkyl, R³ is —R⁵—R⁶, R⁵ is —(CH₂)ₘO—, and m is 2.

In one embodiment of Formula II, R⁶ is pyridyl, R² is lower alkyl, R³ is —R⁵—R⁶, R⁵ is —(CH₂)ₘO—, and m is 2.

In one embodiment of Formula II, R⁶ is phenyl, R² is lower alkyl, R¹ is halogen, R³ is —R⁵—R⁶, R⁵ is —(CH₂)ₘO—, and m is 2.

In one embodiment of Formula II, R⁶ is pyridyl, R² is lower alkyl, R¹ is halogen, R³ is —R⁵—R⁶, R⁵ is —(CH₂)ₘO—, and m is 2.

In one embodiment of Formula II, R² is lower alkyl.

In one embodiment of Formula II, R² is H.

In one embodiment of Formula II, R² is H, R³ is —R⁵—R⁶, R⁵ is —(CH₂)ₘ—, and m is 2.

In one embodiment of Formula II, R² is H, and R⁶ is phenyl.

In one embodiment of Formula II, R² is H, and R⁶ is pyridyl.

In one embodiment of Formula II, R² is H, R⁶ is phenyl, R³ is —R⁵—R⁶, Rd is —(CH₂)ₘ—, and m is 2.

In one embodiment of Formula II, R² is H, R⁶ is pyridyl, R³ is —R⁵—R⁶, R⁵ is —(CH₂)ₘ—, and m is 2.

In one embodiment of Formula II, R³ is —R⁵—R⁶, R⁵ is —(CH₂)ₘ—, and m is 3.

In one embodiment of Formula II, R⁶ is pyrimidine.

In one embodiment of Formula II, R⁶ is pyridazine.

In one embodiment of Formula II, R⁶ is pyrimidine, R³ is —R⁵—R⁶, R⁵ is —(CH₂)ₘ—, and m is 3.

In one embodiment of Formula II, R⁶ is pyridazine, R³ is —R⁵—R⁶, R⁵ is —(CH₂)ₘ—, and m is 3.

In one embodiment of Formula II, R² is lower alkyl, R⁶ is pyrimidine, R³ is —R⁵—R⁶, R⁵ is —(CH₂)ₘ—, and m is 3.

In one embodiment of Formula II, R² is lower alkyl, R⁶ is pyridazine, R³ is —R⁵—R⁶, R⁵ is —(CH₂)ₘ—, and m is 3.

In one embodiment of Formula II, R³ is —R⁵—R⁶, R⁵ is —(CH₂)ₘS—, and m is 1.

In one embodiment of Formula II, R² is H, R³ is —R⁵—R⁶, R⁵ is —(CH₂)ₘS—, and m is 1.

In one embodiment of Formula II, R⁶ is phenyl methylenyl.

In one embodiment of Formula II, R⁶ is phenyl methylenyl, R³ is —R⁵—R⁶, R⁵ is —(CH₂)ₘS—, and m is 1.

In one embodiment of Formula II, R⁶ is phenyl methylenyl, R² is H, R³ is —R⁵—R⁶, R⁵ is —(CH₂)ₘS—, and m is 1.

The application also provides a compound of Formula II selected from the group consisting of:
3-Chloro-5-[3-dimethylamino-2-oxo-5-(3-phenyl-propyl)-1,2-dihydro-pyridin-4-yloxy]-benzonitrile;
3-(3-Bromo-5-ethyl-2-oxo-1,2-dihydro-pyridin-4-yloxy)-5-chloro-benzonitrile;
3-Chloro-5-(5-ethyl-2-oxo-3-vinyl-1,2-dihydro-pyridin-4-yloxy)-benzonitrile;
3-Chloro-5-(3-chloro-5-ethyl-2-oxo-1,2-dihydro-pyridin-4-yloxy)-benzonitrile;
3-(3-Bromo-2-oxo-5-propyl-1,2-dihydro-pyridin-4-yloxy)-5-chloro-benzonitrile;
3-(3-Bromo-5-ethyl-6-methyl-2-oxo-1,2-dihydro-pyridin-4-yloxy)-5-chloro-benzonitrile;
3-[3-Bromo-2-oxo-5-(2-phenoxy-ethyl)-1,2-dihydro-pyridin-4-yloxy]-5-chloro-benzonitrile;
3-(5-Benzyl-3-bromo-2-oxo-1,2-dihydro-pyridin-4-yloxy)-5-chloro-benzonitrile;
3-{3-Bromo-2-oxo-5-[2-(pyridin-3-yloxy)-ethyl]-1,2-dihydro-pyridin-4-yloxy}-5-chloro-benzonitrile;
3-(5-Benzyl-3-bromo-6-methyl-2-oxo-1,2-dihydro-pyridin-4-yloxy)-5-chloro-benzonitrile;
3-[3-Bromo-6-methyl-2-oxo-5-(3-phenyl-propyl)-1,2-dihydro-pyridin-4-yloxy]-5-chloro-benzonitrile;
3-{3-Bromo-2-oxo-5-[2-(pyridin-4-yloxy)-ethyl]-1,2-dihydro-pyridin-4-yloxy}-5-chloro-benzonitrile;
3-Chloro-5-[3-chloro-6-methyl-2-oxo-5-(3-pyridin-4-yl-propyl)-1,2-dihydro-pyridin-4-yloxy]-benzonitrile;
3-Chloro-5-[3-chloro-6-methyl-2-oxo-5-(3-pyridin-2-yl-propyl)-1,2-dihydro-pyridin-4-yloxy]-benzonitrile;
3-[3-Bromo-2-oxo-5-(pyridin-4-ylmethoxymethyl)-1,2-dihydro-pyridin-4-yloxy]-5-chloro-benzonitrile;
3-Chloro-5-[3-chloro-6-methyl-2-oxo-5-(2-phenoxy-ethyl)-1,2-dihydro-pyridin-4-yloxy]-benzonitrile;
3-Chloro-5-[3-chloro-6-methyl-2-oxo-5-(3-pyridin-3-yl-propyl)-1,2-dihydro-pyridin-4-yloxy]-benzonitrile;
3-(5-Benzylsulfanylmethyl-3-bromo-2-oxo-1,2-dihydro-pyridin-4-yloxy)-5-chloro-benzonitrile;
3-Chloro-5-[3-chloro-6-methyl-2-oxo-5-(3-pyrimidin-4-yl-propyl)-1,2-dihydro-pyridin-4-yloxy]-benzonitrile; and
3-Chloro-5-[3-chloro-6-methyl-2-oxo-5-(3-pyridazin-3-yl-propyl)-1,2-dihydro-pyridin-4-yloxy]-benzonitrile.

The application also provides a pharmaceutical composition comprising a compound of Formula I in admixture with at least one pharmaceutically acceptable carrier, diluent or excipient.

The application also provides a pharmaceutical composition comprising the compound of Formula II in admixture with at least one pharmaceutically acceptable carrier, diluent or excipient.

The application also provides a method of treating a disease associated with HIV comprising administering to a patient in need thereof, a therapeutically effective amount of the compound of Formula I.

The application also provides the above method further comprising administering an immune system modulator or an antiviral compound.

The application also provides a method of treating a disease associated with HIV comprising administering to a patient in need thereof, a therapeutically effective amount of the compound of Formula II.

The application also provides the above method further comprising administering an immune system modulator or an antiviral compound.

The application also provides a method for preparing a compound of Formula Ia,

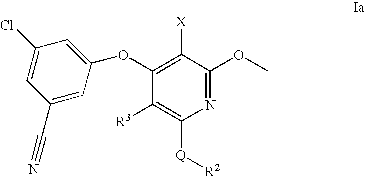

wherein:
X is halide;
Q is Q¹ or Q²;
   Q¹ is lower alkylene;
   Q is Q¹-Q³;
      Q³ is —C(═O)—;
R² is phenyl, heteroaryl, or heterocycloalkyl, optionally substituted with one or more R^{2′};
   R² is lower alkyl or halogen; and
R³ is H, halogen, or lower alkyl;
   comprising the steps of:
a) treating a solution of cupric halide and lithium halide with tert-Butyl nitrite;
b) treating the product of step a) with a compound of Formula Ib;

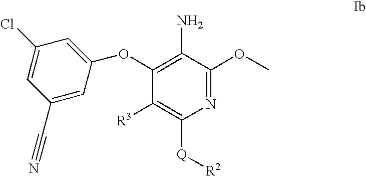

c) treating the product of step b) with an aqueous hydrohalic acid solution.

The application also provides a method for preparing a compound of Formula IIa,

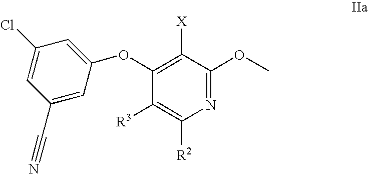

wherein:
R² is H or lower alkyl;
R³ is —R⁴ or —R⁵—R⁶;
   R⁴ is lower alkyl;
   R⁵ is —(CH₂)ₘ—, —(CH₂)ₘO— or —(CH₂)ₘS—;
      m is 1,2, or 3;
   R⁶ is phenyl, phenyl lower alkylenyl, heteroaryl, or heteroaryl lower alkylenyl, optionally substituted with one or more R^{6′}; and
      R^{6′} is lower alkyl, halogen or lower alkoxy; comprising the steps of:
a) treating a solution of cupric halide and lithium halide with tert-Butyl nitrite;
b) treating the product of step a) with a compound of Formula IIb;

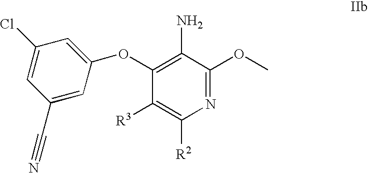

c) adding an aqueous hydrohalic acid solution the product of step b).

### DETAILED DESCRIPTION OF THE INVENTION

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined hereinabove” refers to the first definition provided in the Summary of the Invention.

The term “optional” or “optionally” as used herein means that a subsequently described event or circumstance may, but need not, occur, and that the description includes instances where the event or circumstance occurs and instances in which it does not. For example, “optionally substituted” means that the moiety may be hydrogen or a substituent.

It is contemplated that the definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl refers to either an aryl or a heteroaryl group.

The term “lower alkyl” as used herein denotes an unbranched or branched chain, saturated, monovalent hydrocarbon residue containing 1 to 6 carbon atoms. Examples of alkyl groups include, but are not limited to, lower alkyl groups include methyl, ethyl, propyl, i-propyl, n-butyl, i-butyl, t-butyl or pentyl, isopentyl, neopentyl, hexyl.

The term “haloalkyl” as used herein denotes an unbranched or branched chain alkyl group as defined above wherein 1, 2, 3 or more hydrogen atoms are substituted by a halogen. Examples are 1-fluoromethyl, 1-chloromethyl, 1-bromomethyl, 1-iodomethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl.

The term “aryl” as used herein means a monocyclic or polycyclic-aromatic group comprising carbon and hydrogen atoms. Examples of suitable aryl groups include, but are not limited to, phenyl, tolyl, indenyl, and 1- or 2-naphthyl, as well as benzo-fused carbocyclic moieties such as 5,6,7,8-tetrahydronaphthyl. An aryl group can be unsubstituted or substituted with one or more suitable substituents which substituents include C₁₋₆ alkyl, C₁₋₆ haloalkyl, C₃₋₈ cycloalkyl, C₁₋₆ alkoxy, C₁₋₆ alkylthio, C₁₋₆ alkylsulfinyl, C₁₋₆ sulfonyl, C₁₋₆ haloalkoxy, C₁₋₆ haloalkylthio, halogen, amino, alkylamino, dialkylamino, aminoacyl, acyl, alkoxycarbonyl, carbamoyl, N-alkylcarbamoyl, N,N-dialkylcarbamoyl, nitro and cyano.

A “heteroaryl group” or “heteroaromatic” as used herein means a monocyclic- or polycyclic aromatic ring comprising up to 15 carbon atoms, hydrogen atoms, and one or more heteroatoms, preferably, 1 to 3 heteroatoms, independently selected from nitrogen, oxygen, and sulfur. As well known to those skilled in the art, heteroaryl rings have less aromatic character than their all-carbon counter parts. Thus, for the purposes of the invention, a heteroaryl group need only have some degree of aromatic character.

The term “heterocyclyl” or “heterocycloalkyl” means the monovalent saturated cyclic radical, consisting of one or more rings, preferably one to two rings, of three to eight atoms per ring, incorporating one or more ring heteroatoms (chosen from N, O or S(O)₀₋₂).

The term “alkoxy group” as used herein means an —O-lower alkyl group, wherein alkyl is as defined above such as methoxy, ethoxy, n-propyloxy, i-propyloxy, n-butyloxy, i-butyloxy, t-butyloxy, pentyloxy, hexyloxy, heptyloxy including their isomers.

The term “alkylene” as used herein denotes a divalent linear or branched saturated hydrocarbon radical, having from one to six carbons inclusive, unless otherwise indicated. Examples of alkylene radicals include, but are not limited to, methylene, ethylene, propylene, 2-methyl-propylene, butylene, 2-ethylbutylene.

The term “halogen” as used herein means fluorine, chlorine, bromine, or iodine. Correspondingly, the meaning of the term “halo” encompasses fluoro, chloro, bromo, and iodo.

The term “hydrohalic acid” refers to an acid comprised of hydrogen and a halogen.

The terms “amino”, “alkylamino” and “dialkylamino” as used herein refer to —NH₂, —NHR and —NR₂ respectively and R is alkyl as defined above. The two alkyl groups attached to a nitrogen in a dialkyl moiety can be the same or different. The terms “aminoalkyl”, “alkylaminoalkyl” and “dialkylaminoalkyl” as used herein refer to NH₂(CH₂)n-, RHN(CH₂)n—, and R₂N(CH₂)n- respectively wherein n is 1 to 6 and R is alkyl as defined above

Compounds of formulae I and II which are basic can form pharmaceutically acceptable acid addition salts with inorganic acids such as hydrohalic acids (e.g. hydrochloric acid and hydrobromic acid), sulphuric acid, nitric acid and phosphoric acid, and the like, and with organic acids (e.g. with acetic acid, tartaric acid, succinic acid, fumaric acid, maleic acid, malic acid, salicylic acid, citric acid, methanesulphonic acid and p-toluenesulfonic acid, and the like).

A “prodrug” of a compound of formula (I) herein refers to any compound which releases an active drug according to Formula I in vivo when such prodrug is administered to a mammalian subject. Prodrugs of a compound of Formula I are prepared by modifying one or more functional group(s) present in the compound of Formula I in such a way that the modification(s) may be cleaved in vivo to release the compound of Formula I. Prodrugs include compounds of Formula I wherein a hydroxy, amino, or sulfhydryl group in a compound of Formula I is bonded to any group that may be cleaved in vivo to regenerate the free hydroxyl, amino, or sulfhydryl group, respectively. Examples of produgs include N-acyl-benzenesulfonamide described.

The term “solvate” as used herein means a compound of the invention or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “hydrate” as used herein means a compound of the invention or a salt thereof, that further includes a stoichiometric or non-stoichiometric amount of water bound by non-covalent intermolecular forces.

The term “wild type” as used herein refers to the HIV virus strain which possesses the dominant genotype which naturally occurs in the normal population which has not been exposed to reverse transcriptase inhibitors. The term “wild type reverse transcriptase” used herein has refers to the reverse transcriptase expressed by the wild type strain which has been sequenced and deposited in the SwissProt database with an accession number P03366.

The term “reduced susceptibility” as used herein refers to about a 10 fold, or greater, change in sensitivity of a particular viral isolate compared to the sensitivity exhibited by the wild type virus in the same experimental system.

The term “nucleoside and nucleotide reverse transcriptase inhibitors” (“NRTI”s) as used herein means nucleosides and nucleotides and analogues thereof that inhibit the activity of HIV-1 reverse transcriptase, the enzyme which catalyzes the conversion of viral genomic HIV-1 RNA into proviral HIV-1 DNA.

Typical suitable NRTIs include zidovudine (AZT) available under the RETROVIR tradename; didanosine (ddI) available under the VIDEX tradename.; zalcitabine (ddC) available under the HIVID tradename; stavudine (d4T) available under the ZERIT trademark.; lamivudine (3TC) available under the EPIVIR tradename; abacavir (1592U89) disclosed in WO96/30025 and available under the ZIAGEN trademark; adefovir dipivoxil [bis(POM)-PMEA] available under the PREVON tradename; lobucavir (BMS-180194), a nucleoside reverse transcriptase inhibitor disclosed in EP-0358154 and EP-0736533 and under development by Bristol-Myers Squibb; BCH-10652, a reverse transcriptase inhibitor (in the form of a racemic mixture of BCH-10618 and BCH-10619) under development by Biochem Pharma; emitricitabine [(−)-FTC] licensed from Emory University under U.S. Pat. No. 5,814,639 and under development by Triangle Pharmaceuticals; beta-L-FD4 (also called beta-L-D4C and named beta-L-2′,3′-dicleoxy-5-fluoro-cytidene) licensed by Yale University to Vion Pharmaceuticals; DAPD, the purine nucleoside, (−)-beta-D-2,6,-diamino-purine dioxolane disclosed in EP-0656778 and licensed to Triangle Pharmaceuticals; and lodenosine (FddA), 9-(2,3-dideoxy-2-fluoro-b-D-threo-pentofuranosyl)adenine, an acid stable purine-based reverse transcriptase inhibitor discovered by the NIH and under development by U.S. Bioscience Inc.

The term “non-nucleoside reverse transcriptase inhibitors” (“NNRTI”s) as used herein means non-nucleosides that inhibit the activity of HIV-1 reverse transcriptase.

Typical suitable NNRTIs include nevirapine (BI-RG-587) available under the VIRAMUNE tradename; delaviradine (BHAP, U-90152) available under the RESCRIPTOR tradename; efavirenz (DMP-266) a benzoxazin-2-one disclosed in WO94/03440 and available under the SUSTIVA tradename; PNU-142721, a furopyridine-thio-pyrimide; AG-1549 (formerly Shionogi # S-1153); 5-(3,5-dichlorophenyl)-thio-4-isopropyl-1-(4-pyridyl)methyl-1H-imidazol-2-ylmethyl carbonate disclosed in WO 96/10019; MKC-442 (1-(ethoxy-methyl)-5-(1-methylethyl)-6-(phenylmethyl)-(2,4(1H,3H)-pyrimidinedione); and (+)-calanolide A (NSC-675451) and B, coumarin derivatives disclosed in U.S. Pat. No. 5,489,697.

The term “protease inhibitor” (“PI”) as used herein means inhibitors of the HIV-1 protease, an enzyme required for the proteolytic cleavage of viral polyprotein precursors (e.g., viral GAG and GAG Pol polyproteins), into the individual functional proteins found in infectious HIV-1. HIV protease inhibitors include compounds having a peptidomimetic structure, high molecular weight (7600 daltons) and substantial peptide character, e.g. CRIXIVAN as well as nonpeptide protease inhibitors e.g., VIRACEPT.

Typical suitable PIs include saquinavir available in hard gel capsules under the INVIRASE tradename and as soft gel capsules under the FORTOVASE tradename; ritonavir (ABT-538) available under the NORVIR tradename; indinavir (MK-639) available under the CRIXIVAN tradename; nelfnavir (AG-1343) available under the VIRACEPT; amprenavir (141W94), tradename AGENERASE, a non-peptide protease inhibitor; lasinavir (BMS-234475; originally discovered by Novartis, Basel, Switzerland (CGP-61755); DMP-450, a cyclic urea discovered by Dupont; BMS-2322623, an azapeptide under development by Bristol-Myers Squibb, as a 2nd-generation HIV-1 PI; ABT-378; AG-1549 an orally active imidazole carbamate.

Other antiviral agents include hydroxyurea, ribavirin, IL-2, IL-12, pentafuside and Yissum Project No. 11607. Hydroxyurea (Droxia), a ribonucleoside triphosphate reductase inhibitor, the enzyme involved in the activation of T-cells. Hydroxyurea was shown to have a synergistic effect on the activity of didanosine and has been studied with stavudine. IL-2 is disclosed in Ajinomoto EP-0142268, Takeda EP-0176299, and Chiron U.S. Pat. Nos. RE 33,653, 4,530,787, 4,569,790, 4,604,377, 4,748,234, 4,752,585, and 4,949,314, and is available under the PROLEUKIN (aldesleukin) tradename as a lyophilized powder for IV infusion or sc administration upon reconstitution and dilution with water; a dose of about 1 to about 20 million 1 U/day, sc is preferred; a dose of about 15 million 1 U/day, sc is more preferred. IL-112 is disclosed in WO96/25171 and is available as a dose of about 0.5 microgram/kg/day to about 10 microgram/kg/day, sc is preferred. Pentafiside (DP-178, T-20) a 36-amino acid synthetic peptide, disclosed in U.S. Pat. No. 5,464,933 and available under the FUZEON tradename; pentafuside acts by inhibiting fusion of HIV-1 to target membranes. Pentafuside (3-100 mg/day) is given as a continuous sc infusion or injection together with efavirenz and 2 PI's to HIV-1 positive patients refractory to a triple combination therapy; use of 100 mg/day is preferred. Yissum Project No. 11607, a synthetic protein based on the HIV-1 Vif protein. Ribavirin, 1-.beta.-D-ribofuranosyl-1H-1,2,4-triazole-3-carboxamide, is described in U.S. Pat. No. 4,211,771.

The term “anti-HIV-1 therapy” as used herein means any anti-HIV-1 drug found useful for treating HIV-1 infections in man alone, or as part of multidrug combination therapies, especially the HAART triple and quadruple combination therapies. Typical suitable known anti-HIV-1 therapies include, but are not limited to multidrug combination therapies such as (i) at least three anti-HIV-1 drugs selected from two NRTIs, one PI, a second PI, and one NNRTI; and (ii) at least two anti-HIV-1 drugs selected from NNRTIs and PIs. Typical suitable HAART—multidrug combination therapies include: (a) triple combination therapies such as two NRTIs and one PI; or (b) two NRTIs and one NNRTI; and (c) quadruple combination therapies such as two NRTIs, one PI and a second PI or one NNRTI. In treatment of naive patients, it is preferred to start anti-HIV-1 treatment with the triple combination therapy; the use of two NRTIs and one PI is preferred unless there is intolerance to PIs. Drug compliance is essential. The CD4⁺ and HIV-1-RNA plasma levels should be monitored every 3-6 months. Should viral load plateau, a fourth drug, e.g., one PI or one NNRTI could be added.

Abbreviations used in this application include: acetyl (Ac), acetic acid (HOAc), azo-bis-isobutyrylnitrile (AIBN), 1-N-hydroxybenzotriazole (HOBT), atmospheres (Atm), high pressure liquid chromatography (HPLC), 9-borabicyclo[3.3.1]nonane (9-BBN or BBN), methyl (Me), tert-butoxycarbonyl (Boc), acetonitrile (MeCN), di-tert-butyl pyrocarbonate or boc anhydride (BOC₂O), 1-(3-dimethylaminopropyl)-3-ethylcarbodiimide hydrochloride (EDCI), benzyl (Bn), tert-Butyl nitrite (tBuONO), m-chloroperbenzoic acid (MCPBA), butyl (Bu), methanol (MeOH), benzyloxycarbonyl (cbz or Z), melting point (mp), carbonyl diimidazole (CDI), MeSO₂— (mesyl or Ms), 1,4-diazabicyclo[2.2.2]octane (DABCO), mass spectrum (ms) diethylaminosulfur trifluoride (DAST), methyl t-butyl ether (MTBE), dibenzylideneacetone (Dba), N-carboxyanhydride (NCA), 1,5-diazabicyclo[4.3.0]non-5-ene (DBN), N-bromosuccinimide (NBS), 1,8-diazabicyclo[5.4.0]undec-7-ene (DBU), N-methylpyrrolidone (NMP), 1,2-dichloroethane (DCE), pyridinium chlorochromate (PCC), N,N′-dicyclohexylcarbodiimide (DCC), pyridinium dichromate (PDC), dichloromethane (DCM), propyl (Pr), diethyl azodicarboxylate (DEAD), phenyl (Ph), di-iso-propylazodicarboxylate, DIAD, pounds per square inch (psi), diethyl iso-propylamine (DEIPA), pyridine (pyr), di-iso-butylaluminumhydride, DIBAL-H, room temperature, rt or RT, N,N-dimethyl acetamide (DMA), tert-butyldimethylsilyl or t-BuMe₂Si, (TBDMS), 4-N,N-dimethylaminopyridine (DMAP), triethylamine (Et₃N or TEA), N,N-dimethylformamide (DMF), triflate or CF₃SO₂— (Tf), dimethyl sulfoxide (DMSO), trifluoroacetic acid (TFA), 1,1′-bis-(diphenylphosphino)ethane (dppe), 2,2,6,6-tetramethylheptane-2,6-dione (TMHD), 1,1′-bis-(diphenylphosphino)ferrocene (dppf), thin layer chromatography (TLC), ethyl acetate (EtOAc), tetrahydrofuran (THF), diethyl ether (Et₂O), trimethylsilyl or Me₃Si (TMS), ethyl (Et), p-toluenesulfonic acid monohydrate (TsOH or pTsOH), lithium hexamethyl disilazane (LiHMDS), 4-Me-C₆H₄SO₂— or tosyl (Ts), iso-propyl (i-Pr), N-urethane-N-carboxyanhydride (UNCA), ethanol (EtOH). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### Compounds and Preparation

Examples of representative compounds encompassed by the present invention and within the scope of the invention are contained in the Table 1. The compounds in Table 1 and the preparative examples which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE 1

| Com- | | |
|---|---|---|
| pound | Structure | Nomenclature |
| I-1 | 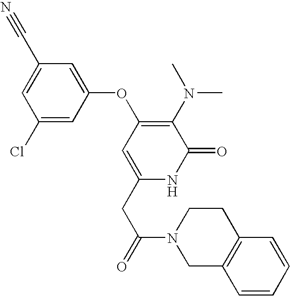 | 3-Chloro-5- {6-[2-(3,4- dihydro-1H- isoquinolin- 2-yl)-2-oxo- ethyl]-3- dimethylamino- 2-oxo- 1,2-dihydro- yloxy}- pyridin-4- benzonitrile |
| I-2 | 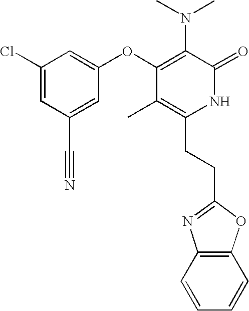 | 3-[6-(2- Benzooxazol- 2-yl-ethyl)- 3- dimethylamino- 5-methyl- 2-oxo-1,2- dihydro- pyridin-4- yloxy]-5- chloro- benzonitrile |
| II-1 | 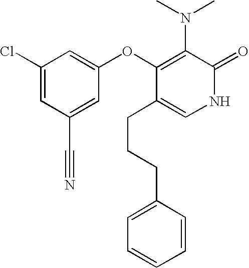 | 3-Chloro-5- [3- dimethylamino- 2-oxo-5- (3-phenyl- propyl)-1,2- dihydro- pyridin-4- yloxy]- benzonitrile |
| I-3 | 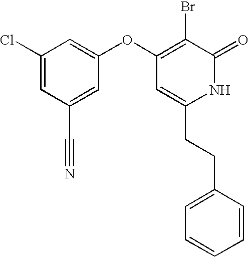 | 3-(3-Bromo- 2-oxo-6- phenethyl- 1,2-dihydro- pyridin-4- yloxy)-5- chloro- benzonitrile |
| I-4 | 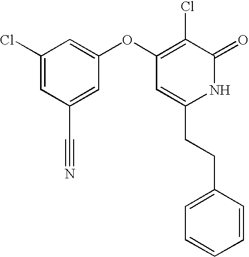 | 3-Chloro-5- (3-chloro-2- oxo-6- phenethyl- 1,2-dihydro- pyridin-4- yloxy)- benzonitrile |
| II-2 | 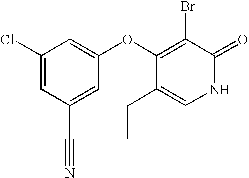 | 3-(3-Bromo- 5-ethyl-2- oxo-1,2- dihydro- pyridin-4- yloxy)-5- chloro- benzonitrile |
| I-5 | 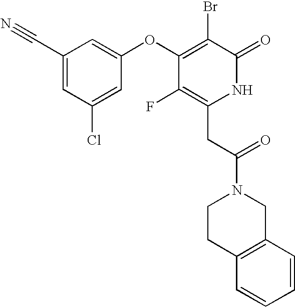 | 3-{3- Bromo-6-[2- (3,4-dihydro- 1H- isoquinolin- 2-yl)-2-oxo- ethyl]-5- fluoro-2-oxo- 1,2-dihydro- pyridin-4- yloxy}-5- chloro- benzonitrile |
| II-3 | 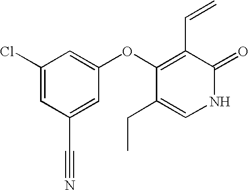 | 3-Chloro-5- (5-ethyl-2- oxo-3-vinyl- 1,2-dihydro- pyridin-4- yloxy)- benzonitrile |
| II-4 | 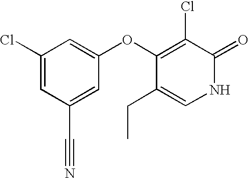 | 3-Chloro-5- (3-chloro-5- ethyl-2-oxo- 1,2-dihydro- pyridin-4- yloxy)- benzonitrile |
| II-5 | 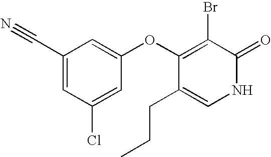 | 3-(3-Bromo- 2-oxo-5- dihydro- pyridin-4- yloxy)-5- chloro- benzonitrile |
| I-6 | 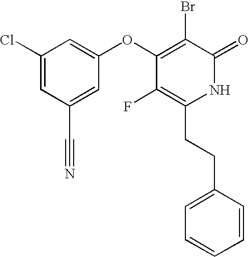 | 3-(3-Bromo- 5-fluoro-2- oxo-6- phenethyl- 1,2-dihydro- pyridin-4- yloxy)-5- chloro- benzonitrile |
| I-7 | 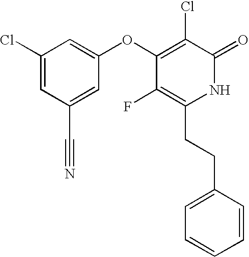 | 3-Chloro-5- (3-chloro-5- fluoro-2-oxo- 6-phenethyl- 1,2-dihydro- pyridin-4- yloxy)- benzonitrile |
| II-6 | 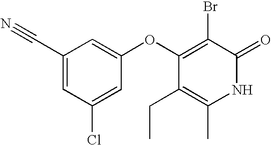 | 3-(3-Bromo- 5-ethyl-6- methyl-2- oxo-1,2- dihydro- pyridin-4- yloxy)-5- chloro- benzonitrile |
| I-8 | 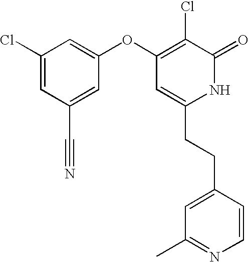 | 3-Chloro-5- {3-chloro-6- [2-(2-methyl- pyridin-4- yl)-ethyl]-2- oxo-1,2- dihydro- pyridin-4- yloxy}- benzonitrile |
| II-7 | 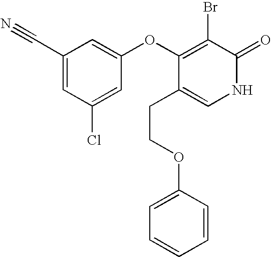 | 3-[3-Bromo- 2-oxo-5-(2- phenoxy- ethyl)-1,2- dihydro- pyridin-4- yloxy]-5- chloro- benzonitrile |
| II-8 | 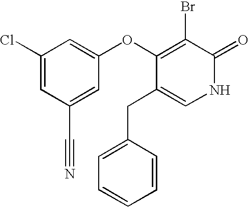 | 3-(5-Benzyl- 3-bromo-2- oxo-1,2- dihydro- pyridin-4- yloxy)-5- chloro- benzonitrile |
| II-9 | 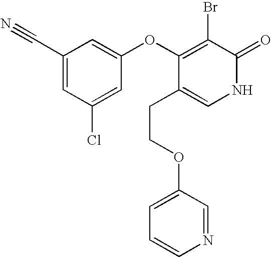 | 3-{3- Bromo-2- oxo-5-[2- (pyridin-3- yloxy)- ethyl]-1,2- dihydro- pyridin-4- yloxy}-5- chloro- benzonitrile |
| I-9 | 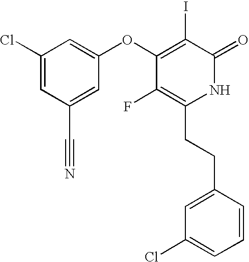 | 3-Chloro-5- {6-[2-(3- chloro- phenyl)- ethyl]-5- fluoro-3- iodo-2-oxo- 1,2-dihydro- pyridin-4- yloxy}- benzonitrile |
| I-10 | 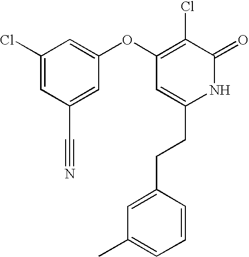 | 3-Chloro-5- [3-chloro-2- oxo-6-(2-m- tolyl-ethyl)- 1,2-dihydro- pyridin-4- yloxy]- benzonitrile |
| I-11 | 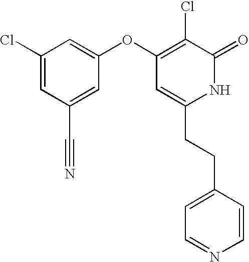 | 3-Chloro-5- [3-chloro-2- oxo-6-(2- pyridin-4-yl- ethyl)-1,2- dihydro- pyridin-4- yloxy]- benzonitrile |
| I-12 | 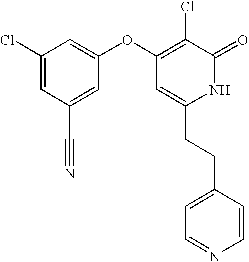 | 3-{3- Bromo-6-[2- (3-chloro- phenyl)- ethyl]-5- fluoro-2-oxo- 1,2-dihydro- pyridin-4- yloxy}-5- chloro- benzonitrile |
| I-13 | 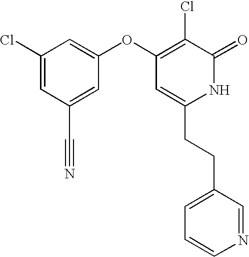 | 3-Chloro-5- [3-chloro-2- oxo-6-(2- pyridin-3-yl- ethyl)-1,2- dihydro- pyridin-4- yloxy]- benzonitrile |
| II-10 | 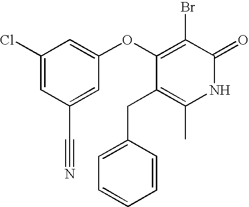 | 3-(5-Benzyl- 3-bromo-6- methyl-2- oxo-1,2- dihydro- pyridin-4- yloxy)-5- chloro- benzonitrile |
| II-11 | 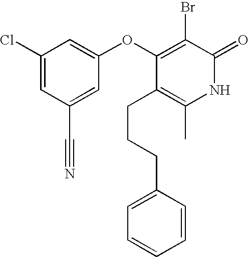 | 3-[3-Bromo- 6-methyl-2- oxo-5-(3- phenyl- propyl)-1,2- dihydro- pyridin-4- yloxy]-5- chloro- benzonitrile |
| I-14 | 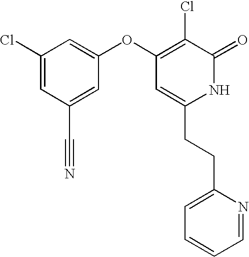 | 3-Chloro-5- [3-chloro-2- oxo-6-(2- pyridin-2-yl- ethyl)-1,2- dihydro- pyridin-4- yloxy]- benzonitrile |
| I-15 | 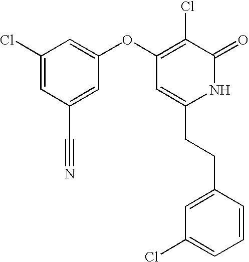 | 3-Chloro-5- {3-chloro-6- [2-(3-chloro- phenyl)- ethyl]-2-oxo- 1,2-dihydro- pyridin-4- yloxyl}- benzonitrile |
| I-16 | 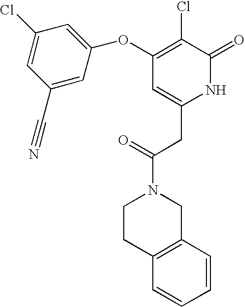 | 3-Chloro-5- {3-chloro-6- [2-(3,4- dihydro-1H- isoquinolin- 2-yl)-2-oxo- ethyl]-2-oxo- 1,2-dihydro- pyridin-4- yloxy}- benzonitrile |
| I-17 | 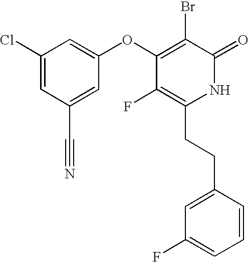 | 3-{3- Bromo-5- fluoro-6-[2- (3-fluoro- phenyl)- ethyl]-2-oxo- 1,2-dihydro- pyridin-4- yloxy}-5- chloro- benzonitrile |
| I-18 | 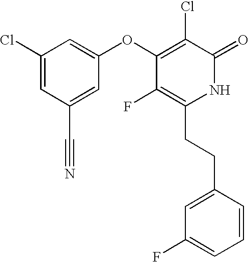 | 3-Chloro-5- {3-chloro-5- fluoro-6-[2- (3-fluoro- phenyl)- ethyl]-2-oxo- 1,2-dihydro- pyridin-4- yloxy}- benzonitrile |
| II-12 | 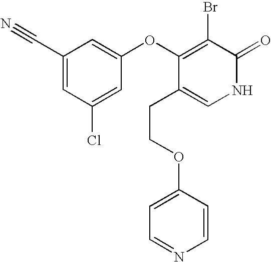 | 3-{3- Bromo-2- oxo-5-[2- (pyridin-4- yloxy)- ethyl]-1,2- dihydro- pyridin-4- yloxy}-5- chloro- benzonitrile |
| II-13 | 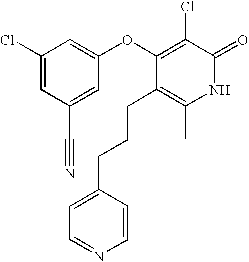 | 3-Chloro-5- [3-chloro-6- methyl-2- oxo-5-(3- pyridin-4-yl- propyl)-1,2- dihydro- pyridin-4- yloxy]- benzonitrile |
| I-19 | 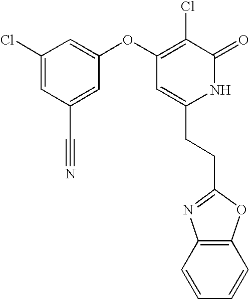 | 3-[6-(2- Benzooxazol- 2-yl-ethyl)- 3-chloro-2- oxo-1,2- dihydro- pyridin-4- yloxy]-5- chloro- benzonitrile |
| II-14 | 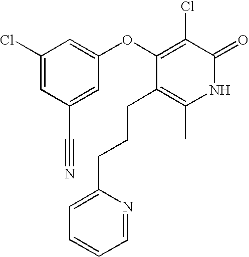 | 3-Chloro-5- [3-chloro-6- methyl-2- oxo-5-(3- pyridin-2-yl- propyl)-1,2- dihydro- pyridin-4- yloxy]- benzonitrile |
| II-15 | 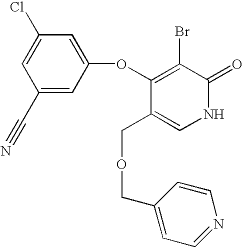 | 3-[3-Bromo- 2-oxo-5- (pyridin-4- ylmethoxy- methyl)-1,2- dihydro- pyridin-4- yloxy]-5- chloro- benzonitrile |
| II-16 | 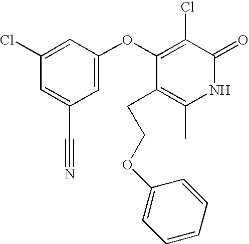 | 3-Chloro-5- [3-chloro-6- methyl-2- oxo-5-(2- phenoxy- ethyl)-1,2- dihydro- pyridin-4- yloxy]- benzonitrile |
| II-17 | 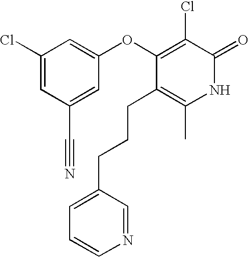 | 3-Chloro-5- [3-chloro-6- methyl-2- oxo-5-(3- pyridin-3-yl- propyl)-1,2- dihydro- pyridin-4- yloxy]- benzonitrile |
| II-18 | 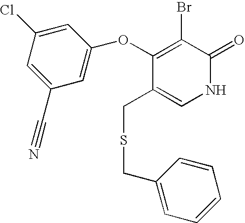 | 3-(5- Benzylsulfanyl- methyl-3- bromo-2- oxo-1,2- dihydro- pyridin-4- yloxy)-5- chloro- benzonitrile |
| II-19 | 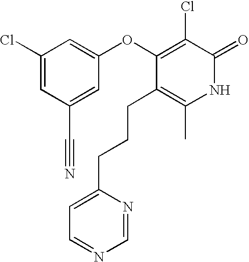 | 3-Chloro-5- [3-chloro-6- methyl-2- oxo-5-(3- pyrimidin-4- yl-propyl)- 1,2-dihydro- pyridin-4- yloxy]- benzonitrile |
| I-20 | 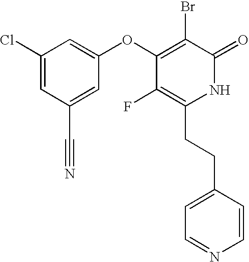 | 3-[3-Bromo- 5-fluoro-2- oxo-6-(2- pyridin-4-yl- ethyl)-1,2- dihydro- pyridin-4- yloxy]-5- chloro- benzonitrile |
| II-20 | 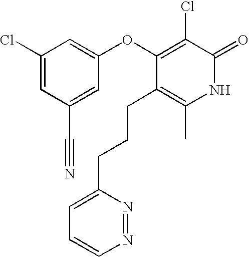 | 3-Chloro-5- [3-chloro-6- methyl-2- oxo-5-(3- pyridazin-3- yl-propyl)- 1,2-dihydro- pyridin-4- yloxy]- benzonitrile |
| I-21 | 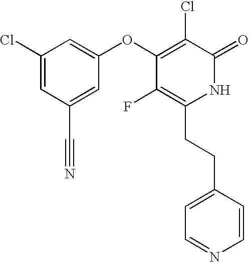 | 3-Chloro-5- [3-chloro-5- fluoro-2-oxo- 6-(2-pyridin- 4-yl-ethyl)- 1,2-dihydro- pyridin-4- yloxy]- benzonitrile |
| I-22 | 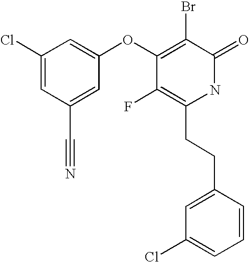 | 3-{3- Bromo-6-[2- (3-chloro- phenyl)- ethyl]-5- fluoro-2-oxo- 1,2-dihydro- pyridin-4- yloxy}-5- chloro- benzonitrile |

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2^{nd} edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

Some compounds in following schemes are depicted with generalized substituents; however, one skilled in the art will immediately appreciate that the nature of the R groups can varied to afford the various compounds contemplated in this invention. Moreover, the reaction conditions are exemplary and alternative conditions are well known. The reaction sequences in the following examples are not meant to limit the scope of the invention as set forth in the claims.

### EXAMPLES

### Example 1

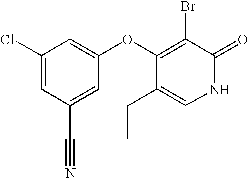

### Prepared According to Literature:

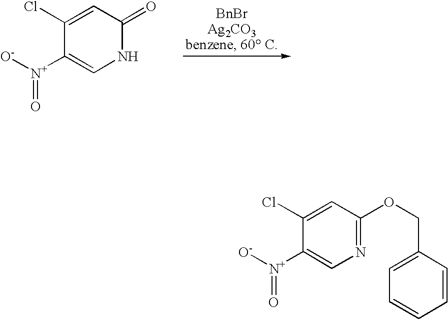

Silver carbonate (5.56 g, 0.51 equiv) and benzyl bromide (5.0 mL, 1.05 equiv) were slowly added to a solution of chloro nitro pyridone (7.00 g, 40.0 mmol) in benzene (135 mL). After heating for 18 hrs at 60° C. the reaction mixture was cooled to rt, filtered over celite, and concentrated in vacuo. The crude residue was then redissolved in EtOAc, washed with water and brine, dried over MgSO₄, and concentrated in vacuo to give crude material (˜4.4 g) that was sufficiently pure to be carried on to the next step.

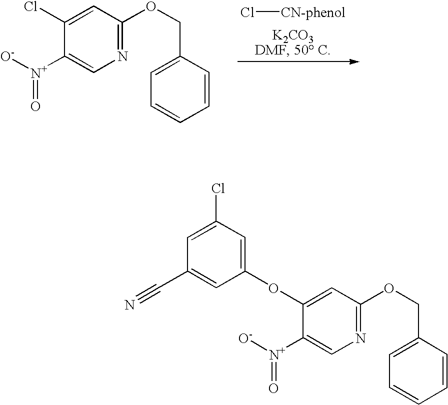

3-chloro-5-cyanophenol (2.74 g, 1.1 equiv) and potassium carbonate (4.93 g, 2.20 equiv) were added to a solution of nitro compound from the previous step (˜4.4 g, ˜16.0 mmol) in DMF (50 mL). After heating for 20 hrs at 50° C. the reaction mixture was cooled to rt, and poured into water (500 mL). The mixture was extracted with ether, washed with water and brine, dried over MgSO₄, and concentrated in vacuo to give crude material (˜7 g) a portion of which was carried on to the next step.

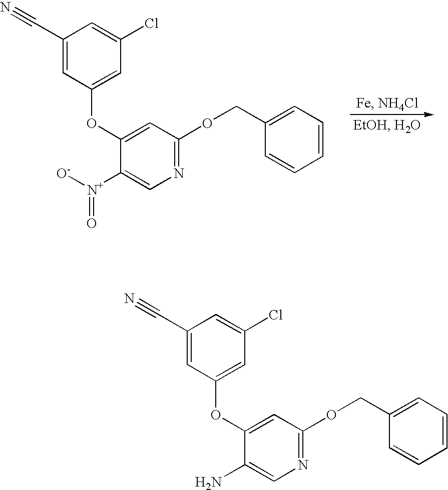

To nitro compound (4.62 g, 12 mmol) suspension in 60 mL of ethanol, NH4Cl (2.54 g, 48 mmol) in H2O (24 mL) was added, followed by Fe (2.68 g, 48 mmol). Heated to 100 C, for 1 h. After cooling to room temp, filtered through celite, the iron residue was washed with ethyl acetate, separated the organic layer, dried over sodium sulfate, concentrated to obtain crude product as light yellow solid 4.1 g, yield 97%.

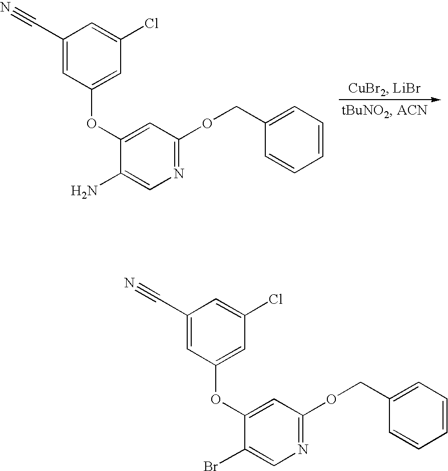

To CuBr2 (2.36 g, 1.2 eq.) LiBr (2.30 g, 1.2 eq) in 60 mL of acetonitrile, at 60 C, tBuNO2 was added, stirred for 15 min, the aniline (3.1 g, 8.8 mmol) in acetonitrile (40 mL) was added. Stirred at 60 C for 2 h. After cooling to room temp, 1% HBr (60 mL) was added to the reaction mixture. Partitioned between ethyl acetate and brine, organic layer washed with water, dried over sodium sulfate, concentrated, purified by silica gel, eluted with hexane:ethyl acetate (9:1), obtained white solid 1.86 g, yield 51%.

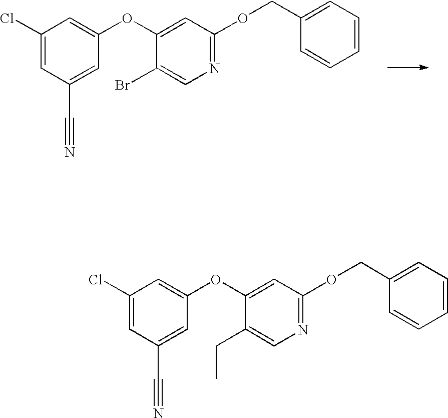

To 3-(2-benzyloxy-5-bromo-pyrodin-4-yloxy)-5-chloro-benzonotrile (1.50 g, 3.61 mmol) and (dppf) PdCl₂ 295 mg, 0.36 mmol) suspended in THF (15 mL), at r.t, diethyl zinc (6.6 mL, 7.22 mmol) was added, followed by dimethylaminoethanol (72 μL, 0.71 mmol), heated to 60° C. for 2 h, quenched the reaction with saturated aqueous ammonium chloride, extracted with ethyl acetate, dried over sodium sulfate, concentrated. Purified by flash column, eluted with 0% to 20% ethyl acetate in Hexane, obtained product 0.61 g, yield 47%.

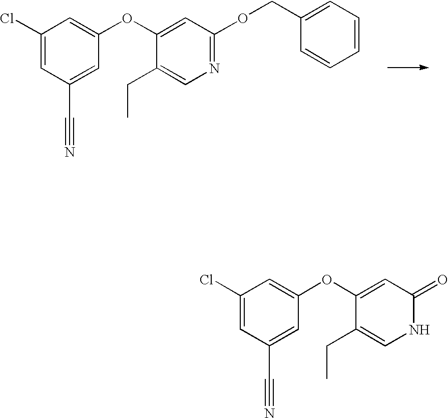

3-(2-benzyloxy-5-ethyl-pyrodin-4-yloxy)-5-chloro-benzonotrile (0.61 g) dissolved in 12 mL of DCM, added TFA (12 mL), heated to 50° C. overnight, removed solvent, Purified by flash column, eluted with 0% to 10% methanol in dichloromethane, obtained product 0.45 g, yield 98%.

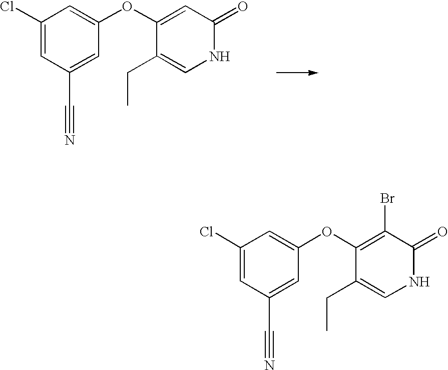

3-Chloro-5-(5-ethyl-2-oxo-1,2-dihydro-pyridin-4-yloxy)-benzonitrile (310 mg, 1.12 mmol) suspended in CH₃CN at r.t, added N-bromo succinimide (1.0 eq) and stirred for 1 h. Removed solvent, purified by flash column, eluted with 10% to 80% ethyl acetate in Hexane, got product as light yellow solid (210 mg), yield 53%.

The following compounds were prepared using the above methods:

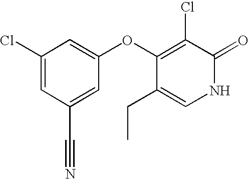

### Example 2

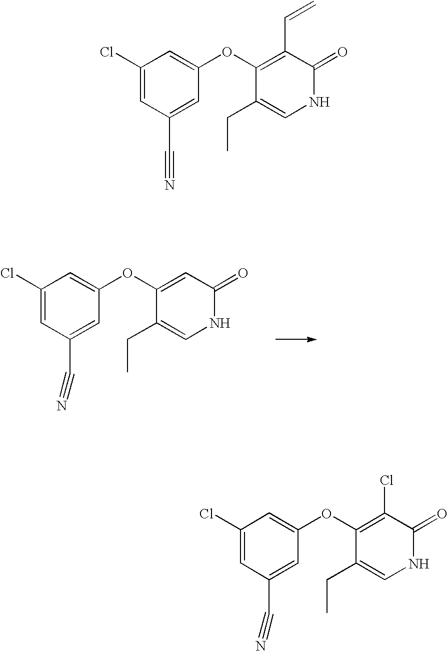

3-Chloro-5-(5-ethyl-2-oxo-1,2-dihydro-pyridin-4-yloxy)-benzonitrile (55 mg, 0.2 mmol) suspended in CH₃CN (1 mL) and isopropanol (1 mL) at r.t, added N-chloro succinimide (1.0 eq) and heated to 60° C. for 2 h. Removed solvent, purified by flash column, eluted with 10% to 80% ethyl acetate in Hexane, got product as off white solid (15.0 mg), yield 24%.

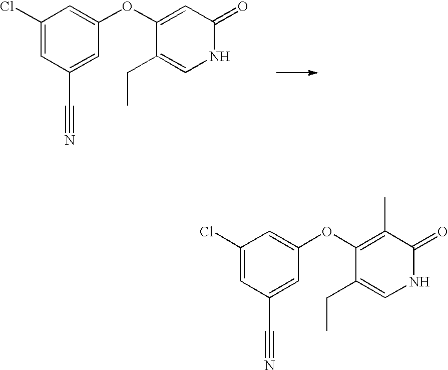

3-Chloro-5-(5-ethyl-2-oxo-1,2-dihydro-pyridin-4-yloxy)-benzonitrile (320 mg, 1.16 mmol) suspended in ethyl acetate (10 mL) and acetic acid (1 mL), added N-iodo succinimide (1.0 eq) and heated to 50° C. for 4 h. Removed solvent, purified by flash column, eluted with 0% to 5% methanol in dichloromethane, got product as light brown solid (180 mg), yield 39%.

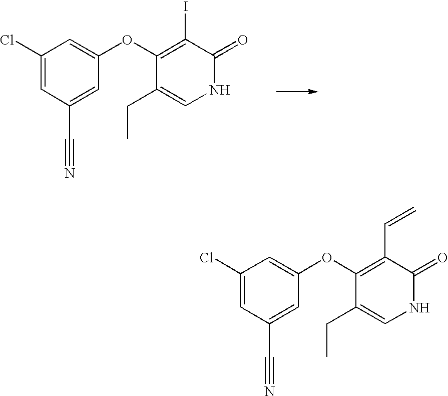

3-Chloro-5-(5-ethyl-3-iodo-2-oxo-1,2-dihydro-pyridin-4-yloxy)-benzonitrile (180 mg, 0.448 mmol) in 3 mL of toluene, under nitrogen atmosphere, added tetrakis (triphenylphosphine) palladium (0) (52 mg, 0.045 mmol), followed by tributyl(vinyl)tin (157 μL, 0.538 mmol), heated to 110° C. for 2 h. Filtered through celite, partitioned between ethyl acetate and brine, organic layer dried over sodium sulfate, concentrated. Purified by flash column, eluted with 0% to 75% ethyl acetate in hexane, obtained white solid 36 mg, yield 27%.

### Example 3

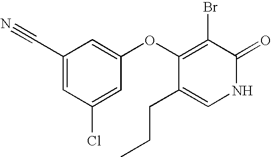

The above compound was prepared as described above substituting allyl tributyltin in the coupling step followed by hydrogenation.

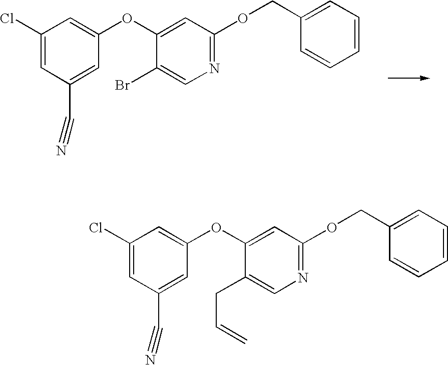

Allyltributyltin (0.637 g, 1.0 equiv) was added to a solution Pd(PPh3)4 (333 mg, 0.15 equiv) and benzyloxypyridine X (800 mg, 1.92 mmol) in DMF (12 mL) and the mixture was heated to 80° C. After 4 h, the mixture was cooled, extracted with ether, washed with water and brine, dried over MgSO₄, and concentrated in vacuo. The resulting mass was chromatographed (SiO₂, 3% to 10% EtOAc/hexanes) to provide allylated product (400 mg, 55%).

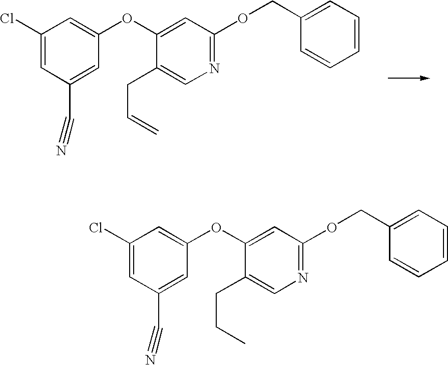

10% Pd/C (14 mg) was added to a solution of the vinyl pyridine (50 mg, 0.13 mmol) in MeOH (1 mL). The mixture was then stirred under an atmosphere of H₂ for 2 h. After which, the mixture was filtered over celite and concentrated in vacuo. Analysis of the crude mass by LC/MS revealed that the material was 90% pure. This material was carried on to the next step without any further purification.

The following compounds were made using this procedure: I-3, I-4, I-14, I-8, I-10, I-15, I-12, I-13, and I-19.

### Example 4

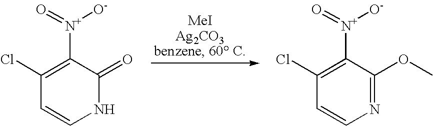

Silver carbonate (9.50 g, 0.60 equiv) and iodomethane (18.0 mL, 5.00 equiv) were slowly added to a solution of chloro nitro pyridone (10.00 g, 56.2 mmol) in benzene (100 mL). After heating for 8 hrs at 50° C. the reaction mixture was cooled to rt, filtered over celite, and concentrated in vacuo. The crude residue was then redissolved in CH₂Cl₂, washed with water and brine, dried over MgSO₄, concentrated in vacuo and chromatographed (SiO2, 100% CH₂Cl₂) to provide methyl ether product (5.81 g, 55%).

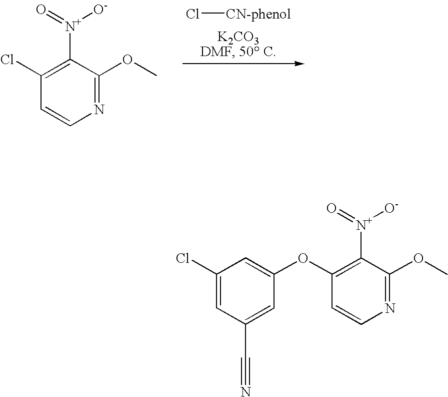

3-chloro-5-cyanophenol (4.73 g, 1.00 equiv) and potassium carbonate (8.52 g, 2.00 equiv) were added to a solution of nitro compound from the previous step (5.81 g, 30.8 mmol) in DMF (90 mL). After heating for 20 hrs at 50° C. the reaction mixture was cooled to rt, and poured into water (500 mL). The mixture was extracted with ether, washed with water and brine, dried over MgSO₄, and concentrated in vacuo to give crude material (˜10 g) which was carried on to the next step.

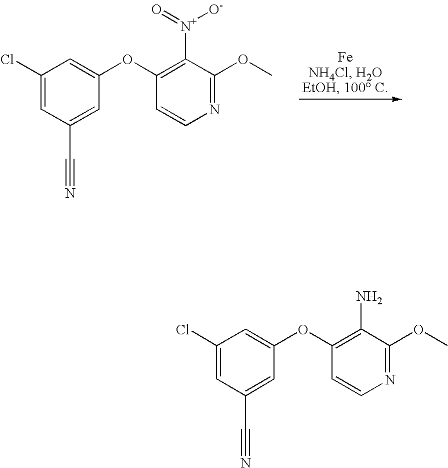

Ammonium chloride (6.59 g, 4.0 equiv) in water (50 mL), and Fe powder (6.88 g, 4.0 equiv) were slowly added to a solution of nitro compound from the previous step (˜10 g, ˜30.8 mmol) in EtOH (150 mL). After heating for 2 h at 100° C. the reaction mixture was filtered hot over celite, and the residual iron washed with EtOAc. The organic layers were then washed with water and brine, dried over MgSO₄, and concentrated in vacuo to provide aniline product (8.26 g, 97% over two steps).

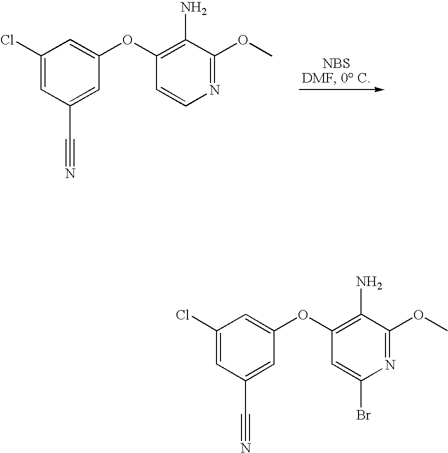

NBS (3.72 g, 1.05 equiv) was added in one portion to a solution of aniline compound from the previous step (5.43 mg, 19.7 mmol) in DMF (100 mL) at 0° C. After stirring from 0° C. to room temperature over 2.5 hrs reaction mixture was poured into water. The mixture was extracted with ether, washed with water and brine, dried over MgSO₄, concentrated in vacuo to give ˜6.5 g aniline product that was sufficiently pure to be carried on. Alternatively, this material can be chromatographed (SiO2, 10% to 50% EtOAc/hexanes).

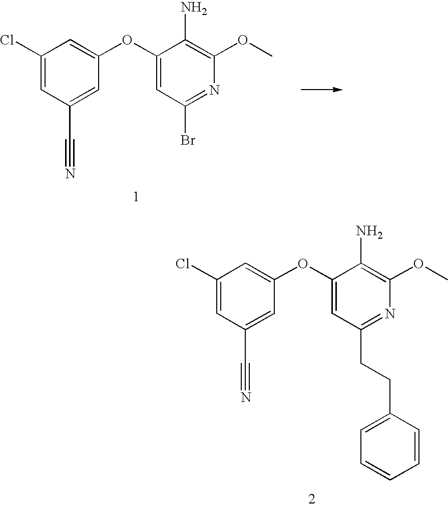

To a solution of 6-Bromo-pyridone 1 (500 mg, 1.41007 mmol) in 1,4-Dioxane (anhydrous, 15 mL) was added Bis(tri-t-butylphosphine)palladium (0) (Strem, 108.09 mg, 0.21151 mmol, 0.15 eq.) followed by Phenethylzinc bromide (Rieke, 0.5M solution in THF, 4.230 mL, 2.11510 mmol, 1.5 eq.) and the reaction mixture stirred under Argon at room temperature overnight. Purification by silica flash column chromatography (Hexane/Ethyl acetate 5 to 40%) gave 335.30 mg (62.6%) of compound 2 as a yellow oil.

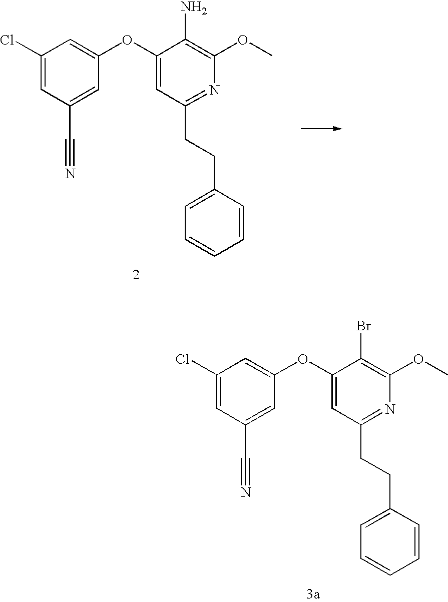

Lithium bromide (150.89 mg, 1.73753 mmol, 3 eq.) and Copper(II) bromide (99%, 156.80 mg, 0.69501 mmol, 1.2 eq.) in Acetonitrile (anhydrous, 1.0 mL) were stirred at 60° C. for a few minutes, then tert-Butyl nitrite (90%, 135.5 μL, 1.02514 mmol, 1.77 eq.) added, stirred for 10 min at 60° C., and eventually a solution of 2 (220 mg, 0.57918 mmol) in Acetonitrile (anhydrous, 2.0 mL) added. The reaction mixture was stirred at 60° C. for 2 h, then cooled to 0° C. and quenched with dil. Hydrobromic acid. Extracted with Ethyl acetate. The combined extracts were washed with sat. aq. Sodium chloride solution, dried over Magnesium sulfate and the solvent evaporated under reduced pressure.

Purification by silica flash column chromatography (Hexane/Dichloromethane 7 to 60%) gave 140.76 mg (54.8%) of compound 3a as a white solid.

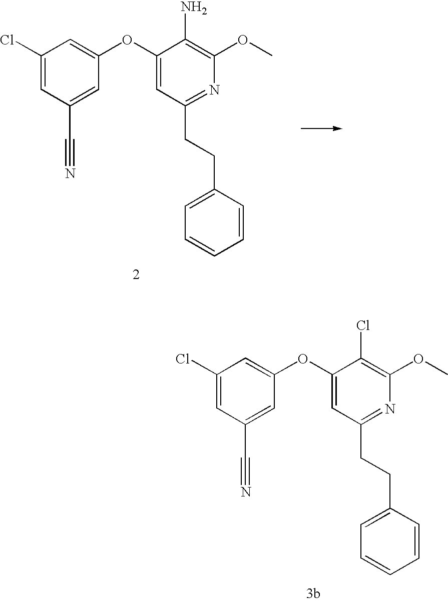

3b was prepared analogous to 3a (see above). Lithium chloride and Copper(II) chloride were used instead of Lithium bromide and Copper(II) bromide respectively. Isolated 65.03 mg (56.2%) of compound 3b as a white solid.

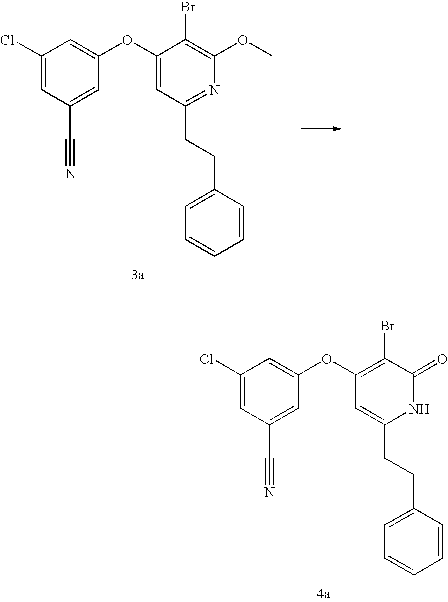

3a (50 mg, 0.11268 mmol) was dissolved in Acetonitrile (anhydrous, 5 mL), Sodium iodide (67.56 mg, 0.45072 mmol, 4 eq.) added, then Chlorotrimethylsilane (57.2 μL, 0.45072 mmol, 4 eq.) added dropwise. The reaction mixture was stirred for 20 min at room temperature. Methanol with Triethylamine (excess) was added to the reaction mixture. Concentrated under reduced pressure.

Absorbed onto silica gel (700 mg). Purification by silica flash column chromatography (Dichloromethane/Methanol 1 to 10%) gave 14.10 mg (29.1%) of compound 4a as a white crystalline solid.

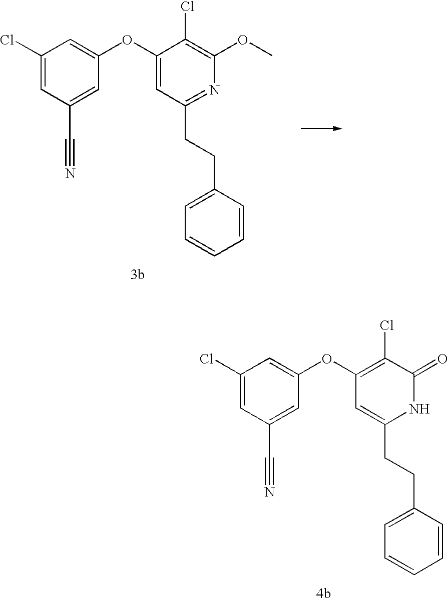

3b (62.23 mg, 0.15586 mmol) was dissolved in Acetonitrile (anhydrous, 5 mL), Sodium iodide (93.45 mg, 0.62342 mmol, 4 eq.) added, then Chlorotrimethylsilane (79.1 μL, 0.62342 mmol, 4 eq.) added dropwise. The reaction mixture was stirred for 10 min at room temperature. Methanol with Triethylamine (excess) was added to the reaction mixture. Concentrated under reduced pressure.

Absorbed onto silica gel (700 mg). Purification by silica flash column chromatography (Dichloromethane/Methanol 1 to 10%) gave 31.12 mg (51.8%) of compound 4b as a white crystalline solid.

### Example 5

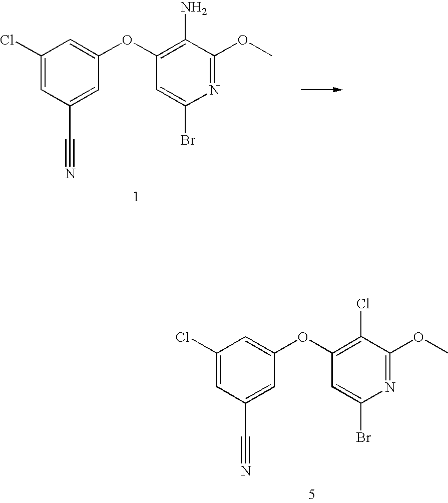

Lithium chloride (1271.76 mg, 30 mmol, 3 eq.) and Copper(II) chloride (97%, 1663.32 mg, 12 mmol, 1.2 eq.) in Acetonitrile (anhydrous, 15 mL) were stirred at 60° C. for a few minutes, then tert-Butyl nitrite (90%, 2339.2 μL, 17.7 mmol, 1.77 eq.) added, stirred for 10 min at 60° C., and eventually a solution of 1 (3545.93 mg, 10 mmol) in Acetonitrile (anhydrous, 35 mL) added. The reaction mixture was stirred at 60° C. for 2 h, then cooled to 0° C. and quenched with dil. Hydrochloric acid. Extracted with Ethyl acetate. The combined extracts were washed with sat. aq. Sodium chloride solution, dried over Magnesium sulfate and the solvent evaporated under reduced pressure. Purification by silica flash column chromatography (Hexane/Ethyl acetate 2 to 20%) gave 2373.6 mg (63.5%) of compound 5 as a light yellow solid.

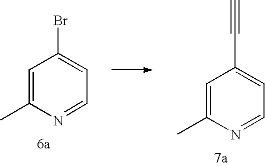

To 4-Bromo-2-methylpyridine (6a, 500 mg, 2.90655 mmol), Copper(I) iodide (55.35 mg, 0.29065 mmol, 0.1 eq.) and trans-Dichlorobis(triphenylphosphine)palladium(II) (Strem, 99%, 206.07 mg, 0.29065 mmol. 0.1 eq.) in Triethylamine (10.5 mL) was added Trimethylsilylacetylene (98%, 503 μL, 3.48786 mmol, 1.2 eq.) and the reaction mixture stirred under Nitrogen at room temperature overnight. Added sat. aq. Sodium chloride solution to the reaction mixture, extracted with Ethyl acetate. The combined extracts were washed with sat. aq. Sodium chloride solution, dried over Magnesium sulfate, and the solvent evaporated under reduced pressure.

The residue thus obtained was taken up in THF (35 mL), the solution cooled to 0° C., and Tetrabutylammonium fluoride (3.2 ml of a 1.0M solution in THF, approx. 1.1 eq.) added. After stirring 15 min at 0° C., sat. aq. Sodium chloride solution was added and the mixture extracted with Ethyl acetate. The combined org. extracts were washed with sat. aq. Sodium chloride solution, dried over Magnesium sulfate, and the solvent evaporated under reduced pressure.

Purification by silica flash column chromatography (Hexane/Ethyl acetate 12 to 100%) gave 184.20 mg (43.3%) of compound 7a as a brown solid.

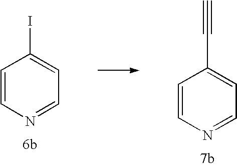

7b was prepared analogous to 7a (see above). Isolated 23.83 mg (11.6%) of compound 7b as a light brown solid.

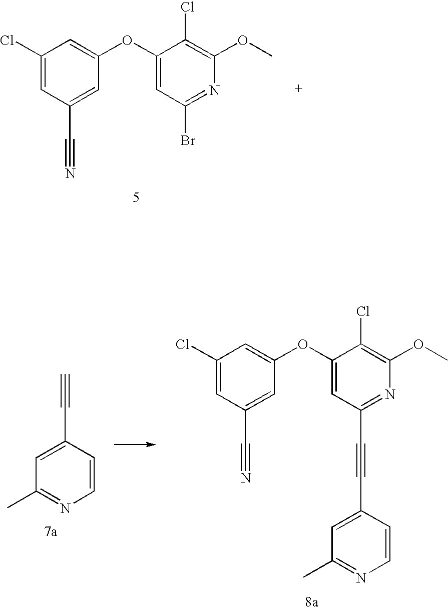

To 5 (50 mg, 0.13368 mmol), Copper(I) iodide (2.55 mg, 0.01337 mmol, 0.1 eq.) and trans-Dichlorobis(triphenylphosphine)palladium(II) (Strem, 99%, 9.48 mg, 0.01337 mmol. 0.1 eq.) in Triethylamine (0.5 mL) was added acetylene 7a (23.49 mg, 0.16042 mmol, 1.2 eq.) and the reaction mixture stirred under Nitrogen at room temperature overnight. Added sat. aq. Sodium chloride solution to the reaction mixture, extracted with Ethyl acetate. The combined extracts were washed with sat. aq. Sodium chloride solution, dried over Magnesium sulfate, and the solvent evaporated under reduced pressure.

Purification by silica flash column chromatography (Hexane/Ethyl acetate 10 to 80%) gave 23.73 mg (43.3%) of compound 8a.

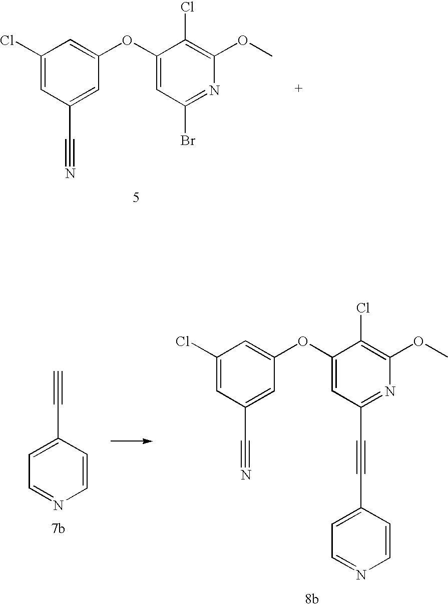

8b was prepared analogous to 8a (see above). Isolated 38.51 mg (42.1%) of compound 8b as a light yellow solid.

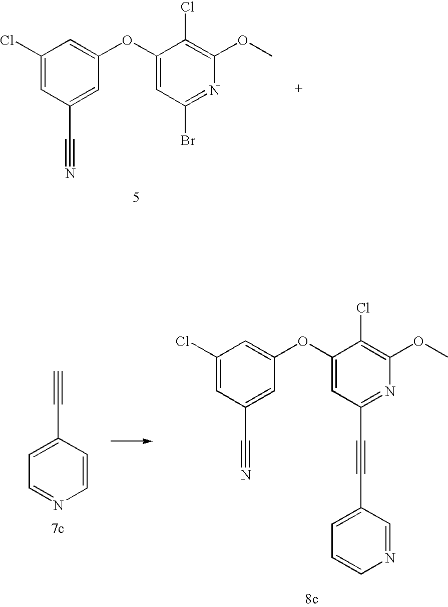

8c was prepared analogous to 8a (see above). [7c is commercially available] Isolated 68.00 mg (57.2%) of compound 8c as a white solid.

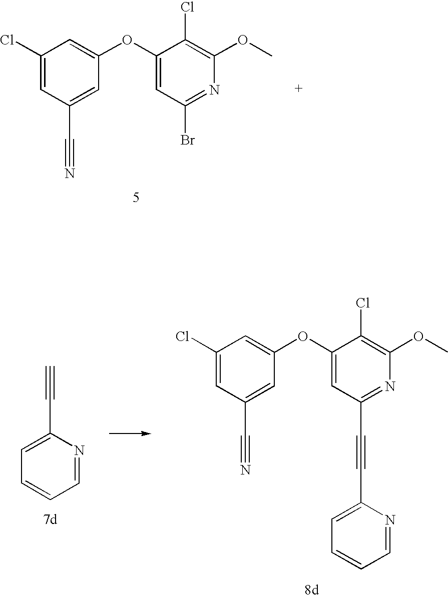

8d was prepared analogous to 8a (see above). [7d is commercially available] Isolated 50.20 mg (42.2%) of compound 8d as a white solid.

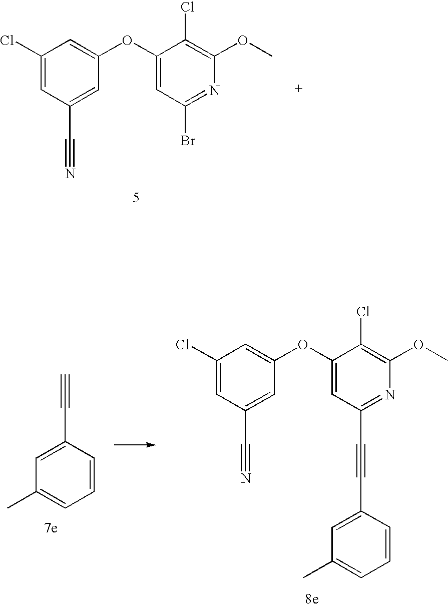

8e was prepared analogous to 8a (see above). [7e is commercially available] Isolated 58.71 mg (71.7%) of compound 8e.

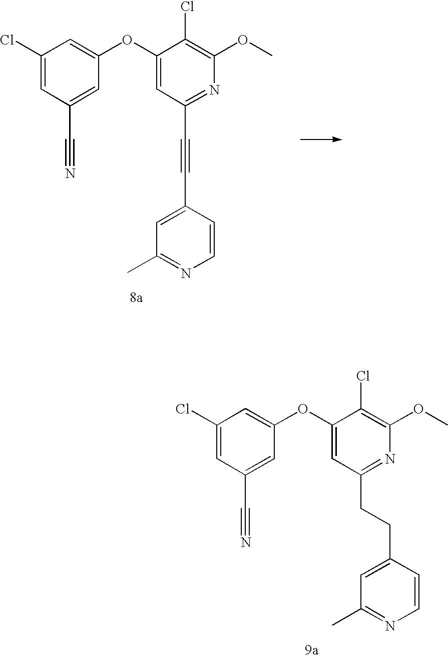

A solution of 8a (22.73 mg, 0.0554 mmol) in THF (2.7 mL) with Palladium on carbon (10 wt. %, 7.60 mg) was stirred under a Hydrogen atmosphere (balloon) at room temperature until LC/MS analysis indicated virtually complete conversion (60 min). Filtered off the catalyst through a membrane filter and evaporated the solvent under reduced pressure.

Purification by silica flash column chromatography (Hexane/Ethyl acetate 12 to 100%) gave 14.54 mg (63.3%) of compound 9a.

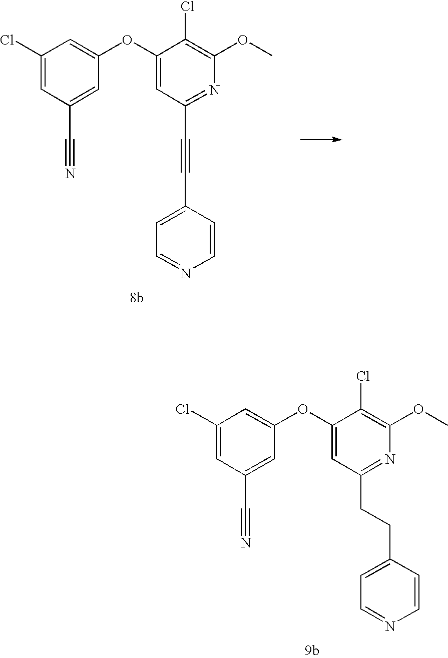

9b was prepared analogous to 9a (see above). Isolated 29.16 mg (77.0%) of compound 9b.

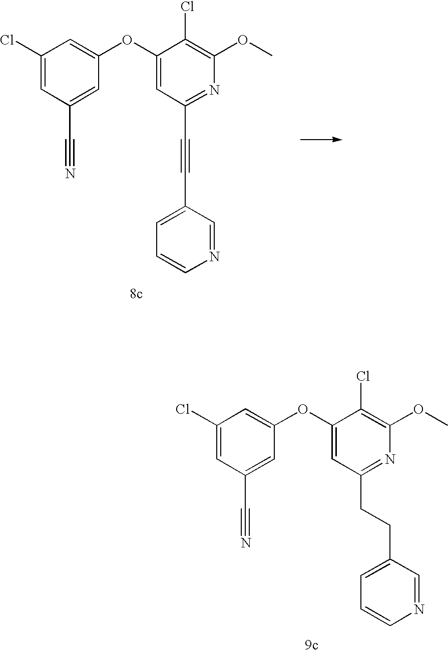

9c was prepared analogous to 9a (see above). Isolated 53.26 mg (77.5%) of compound 9c as a white solid.

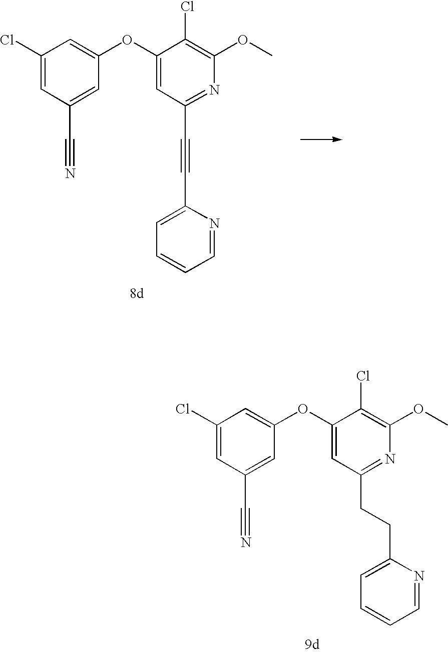

9d was prepared analogous to 9a (see above). Isolated 34.27 mg (67.6%) of compound 9d as a white solid.

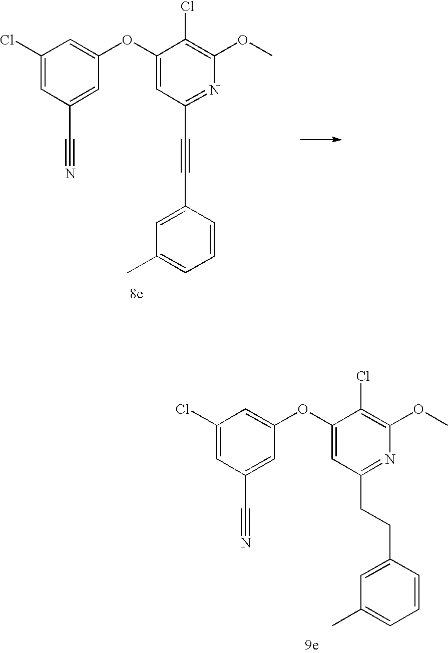

9e was prepared analogous to 9a (see above). Isolated 49.90 mg (84.2%) of compound 9e as a white solid.

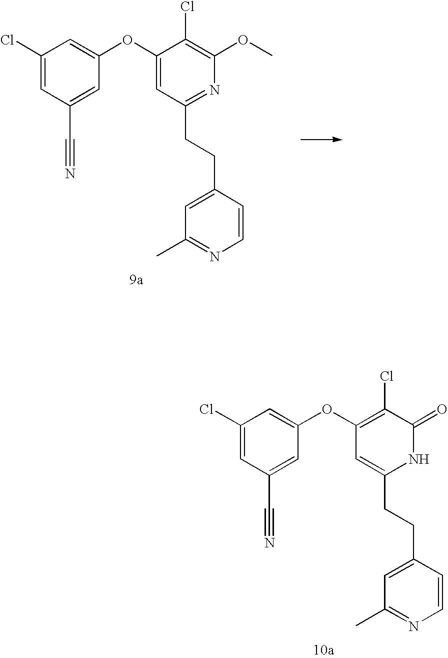

To a solution of 9a (22.91 mg, 0.05530 mmol) in Acetonitrile (anhydrous, 3.0 mL) was added Sodium iodide (20.72 mg, 0.13825 mmol, 2.5 eq.) followed by a solution of Chlorotrimethylsilane (99%, 17.7 μL, 0.13825 mmol, 2.5 eq.) in Acetonitrile (anhydrous, 0.5 mL). Stirred at room temperature for 3 h. Added a mixture of sat. aq. Sodium chloride solution (2 mL) and sat. aq. Sodium hydrogencarbonate (1.0 mL) solution, and extracted with Ethyl acetate. The combined extracts were dried over Magnesium sulfate, and the solvent evaporated under reduced pressure. Purification by prep. TLC (Dichloromethane/Methanol 5%) gave 13.95 mg (40.6%) of compound 10a as a white solid.

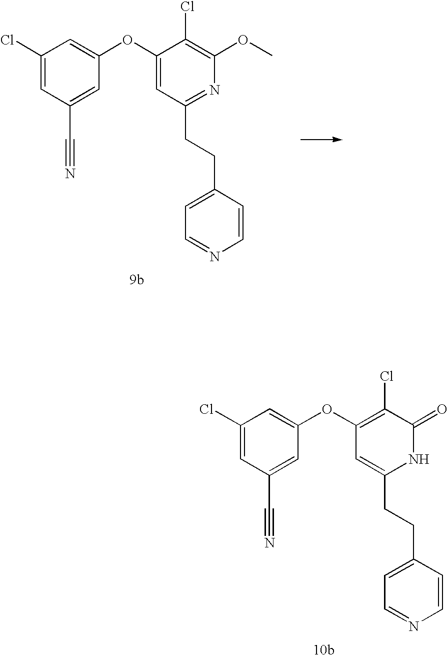

10b was prepared analogous to 10a (see above). The crude product was purified by silica flash column chromatography (Dichloromethane/Methanol 0 to 10%) instead of prep. TLC. Isolated 16.85 mg (59.9%) of compound 10b as a white solid.

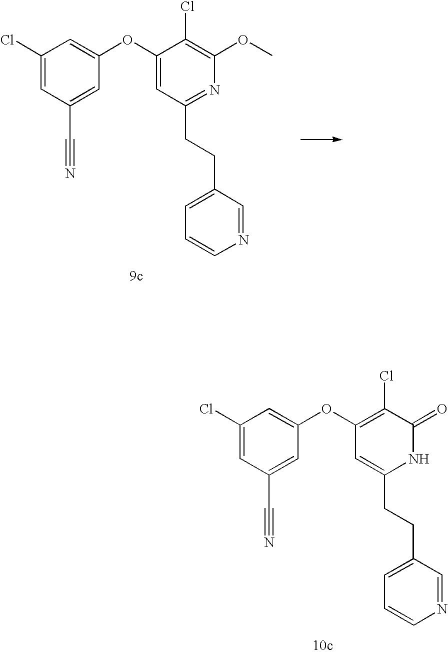

10c was prepared analogous to 10a (see above). Isolated 4.50 mg (8.8%) of compound 10c as a light brown solid.

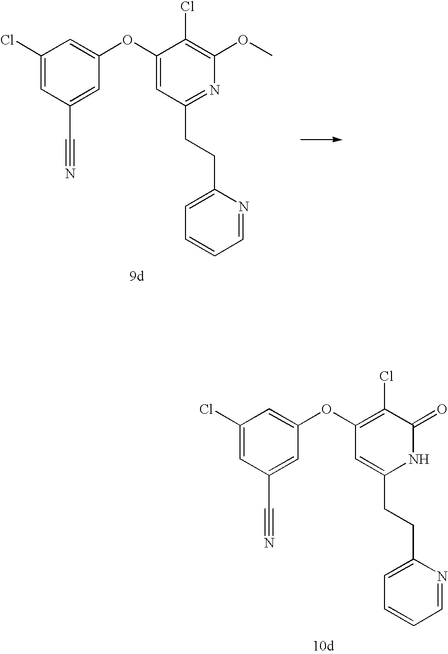

10d was prepared analogous to 10a (see above). The crude product was purified by trituration with Methanol instead of prep. TLC. Isolated 6.62 mg (20.0%) of compound 10d as a light brown solid.

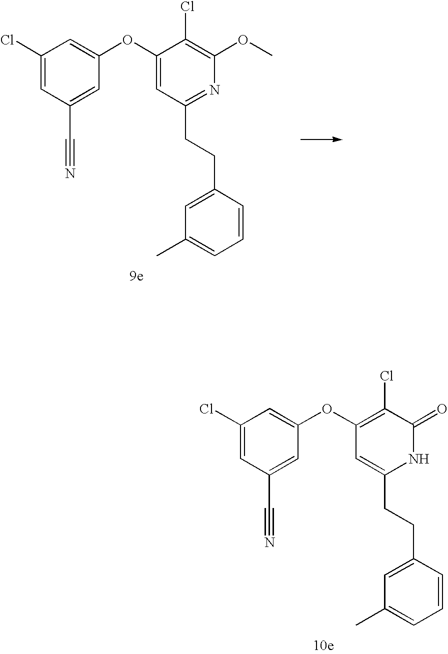

10e was prepared analogous to 10a (see above). The crude product was purified by trituration with Methanol instead of prep. TLC. solated 9.40 mg (19.6%) of compound 10e as a white solid.

### Example 6

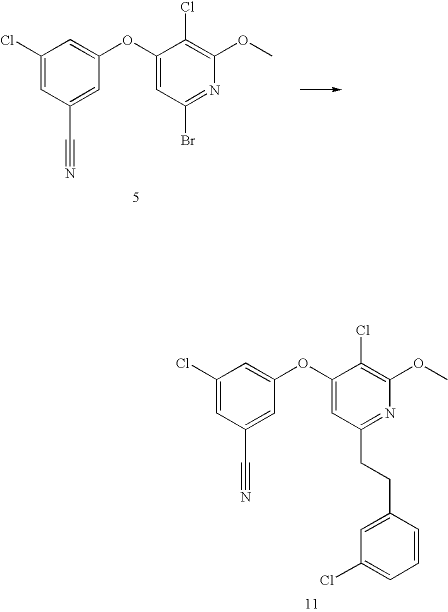

To a solution of 6-Bromo-pyridone 5 (112.21 mg, 0.3 mmol) in 1,4-Dioxane (anhydrous, 4.8 mL) was added Bis(tri-t-butylphosphine)palladium (0) (Strem, 23.47 mg, 0.045 mmol, 0.15 eq.) followed by 3-Chlorophenethylzinc bromide (Rieke, 0.5M solution in THF, 900 μL, 0.45 mmol, 1.5 eq.) and the reaction mixture stirred under Argon at room temperature overnight. Concentrated the reaction mixture under reduced pressure. Purification by silica flash column chromatography (Hexane/Ethyl acetate 2 to 20%) gave 74.17 mg (57.0%) of compound 11 as a white solid.

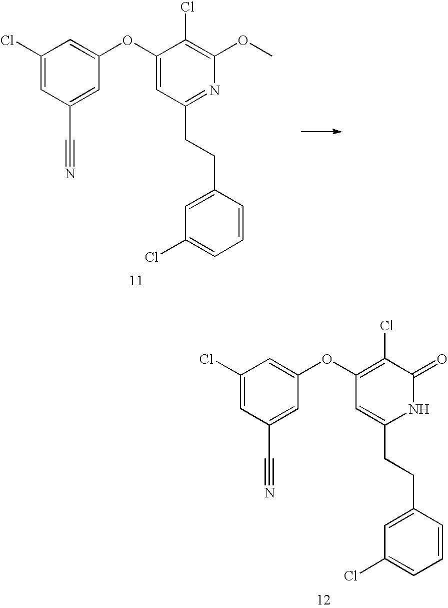

To a solution of 11 (74.17 mg, 0.17101 mmol) in Acetonitrile (anhydrous, 9.7 mL) was added Sodium iodide (64.08 mg, 0.42752 mmol, 2.5 eq.) followed by a solution of Chlorotrimethylsilane (99%, 54.6 μL, 0.42752 mmol, 2.5 eq.) in Acetonitrile (anhydrous, 1.1 mL). Stirred at room temperature for 2 h. Added a mixture of sat. aq. Sodium chloride solution and sat. aq. Sodium hydrogencarbonate solution, and extracted with Ethyl acetate. The combined extracts were dried over Magnesium sulfate, and the solvent evaporated under reduced pressure.

The residue thus obtained was triturated with Methanol and warm (60° C.) Acetonitrile to give 25.70 mg (35.8%) of compound 12 as a white solid.

### Part 4

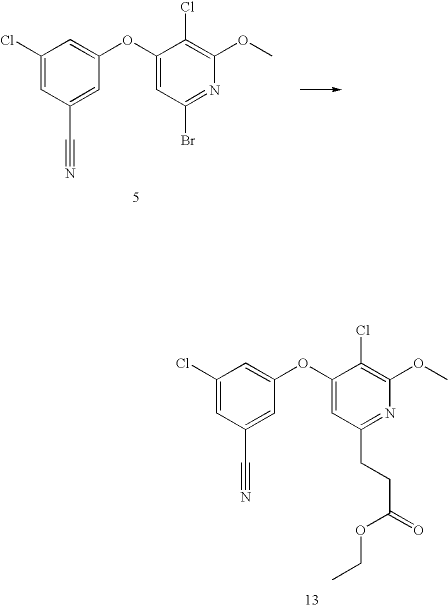

To a solution of 6-Bromo-pyridone 5 (561.03 mg, 1.5 mmol) in 1,4-Dioxane (anhydrous, 24 mL) was added Bis(tri-t-butylphosphine)palladium (0) (Strem, 117.33 mg, 0.225 mmol, 0.15 eq.) followed by 3-Ethoxy-3-oxopropylzinc bromide (Rieke, 0.5M solution in THF, 4.5 mL, 2.25 mmol, 1.5 eq.) and the reaction mixture stirred under Argon at room temperature overnight. Concentrated the reaction mixture under reduced pressure. Purification by silica flash column chromatography (Hexane/Ethyl acetate 2 to 20%, 2^{nd} run 1 to 10%) gave 430.01 mg (72.5%) of compound 13 as a white solid.

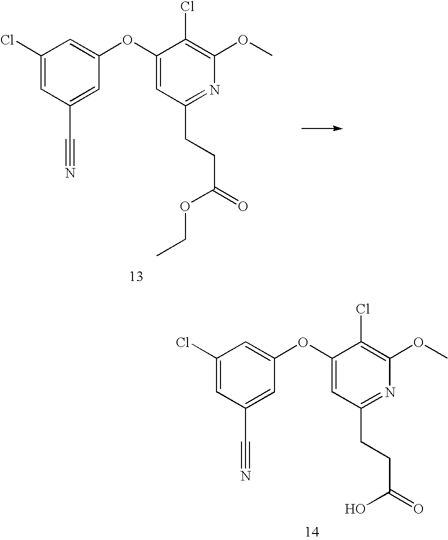

To a solution of 13 (428.34 mg, 1.08373 mmol) in THF (17.0 mL) was added a solution of Lithium hydroxide (264.81 mg, 10.83732 mmol, 10 eq.) in Water (4.2 mL) and the reaction mixture stirred at room temperature for 8 h. The reaction mixture was then diluted with Water and Ethyl acetate, acidified with 1M Hydrochloric acid, the org. phase was washed with sat. aq. Sodium chloride solution, dried over Magnesium sulfate, and the solvent evaporated under reduced pressure. Purification by silica flash column chromatography (Dichloromethane/Methanol 1 to 8%, 2^{nd} run 0.5 to 5%) gave 216.64 mg (54.4%) of compound 14 as a white solid.

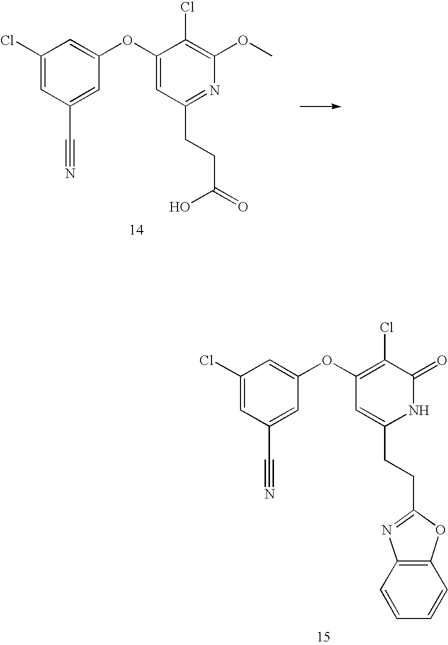

To a suspension of 14 (36.72 mg, 0.1 mmol) in Acetonitrile (anhydrous, 1.5 mL) was added 2-Aminophenol (99%, 11.02 mg, 0.1 mmol, 1 eq.), polymer supported triphenylphosphine (Polymer Laboratories PL-TPP Resin, loading 1.5 mmol/g, 200 mg, 0.3 mmol, 3 eq.) and Trichloroacetonitrile (98%, 20.5 μL, 0.2 mmol, 2 eq.) sequentially. The reaction mixture was heated in the microwave (Personal Chemistry Emrys Optimizer EXP) to 150° C. for 15 min. After cooling the resin was filtered off and washed with Dichloromethane/Methanol (8:2) and Methanol. The combined filtrates were concentrated under reduced pressure.

The crude material thus obtained was triturated with warm (60° C.) Acetonitrile. Purification by prep. HPLC gave 6.41 mg (15.0%) of compound 15 as a white solid.

### Example 7

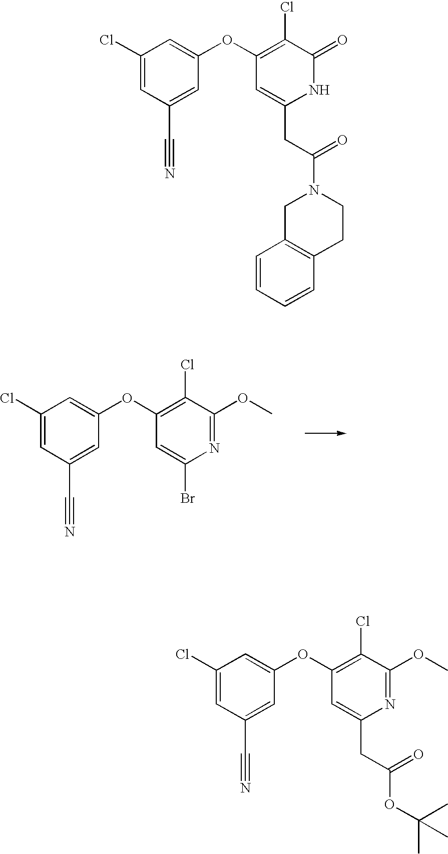

2-tert-Butoxy-2-oxoethylzinc chloride (6.42 mL, 0.5M, 1.2 equiv) was added to a solution of bis(tritertbutylphosphine) palladium (137 mg, 0.10 equiv) and bromide s.m. (999 mg, 2.67 mmol) in dioxane (18 mL) at rt. This solution was then stirred at rt overnight, after which the mixture was quenched with sat. NH₄Cl. The mixture was then extracted with EtOAc, washed with water and brine, dried over MgSO₄, concentrated in vacuo, and chromatographed (SiO₂, 5% to 25% EtOAc/hexanes) to provide coupled product (825 mg, 75%).

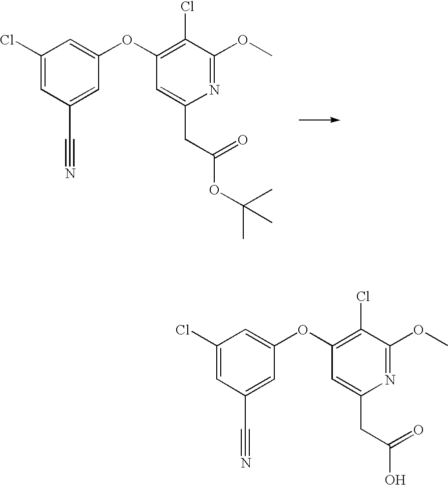

TFA (2 mL) was added to a solution ester (800 mg, 1.96 mmol) in DCM (6 mL) at rt. This solution was stirred at rt for 4 h, after which the mixture was concentrated in vacuo. Toluene (6 mL) was then added and the mixture was further concentrated to provide phenyl acetic acid product (690 mg, 99%).

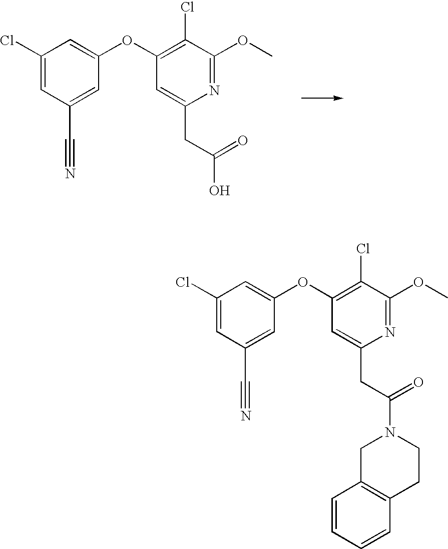

EDCI (54 mg, 1.7 equiv) was added to a solution of 1,2,3,4-tetrahydroisoquinoline (29 mg, 1.3 equiv), Hunig's base (26 mg, 1.2 equiv), HOBT (25 mg, 1 equiv), DMAP (20 mg, 1 equiv), and phenyl acetic acid (59 mg, 0.17 mmol) in DMF (2 mL) at rt. This mixture was then stirred at rt overnight, after which a tan precipitate had formed. This material was filtered and washed with water to provide pure amide product (77 mg, 99%).

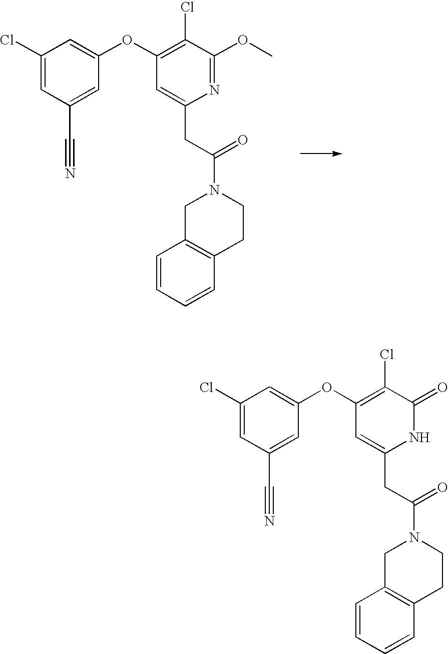

TMSCl (52 μL, 2.5 equiv) was slowly added to a solution of NaI (61 mg, 2.5 equiv) and the amide (76 mg, 0.16 mmol) in acetonitrile (10 mL). After stirring for 3 h at rt an orange precipitate had formed, to this was added water (25 mL). The resulting mixture was filtered, washed with water and EtOAc to give to provide the resulting pyridone (34 mg, 46%).

### Example 6

The following compounds were prepared according to the general

procedure below: I-6, I-7, I-9, I-22, I-17, and I-18.

### Step 1

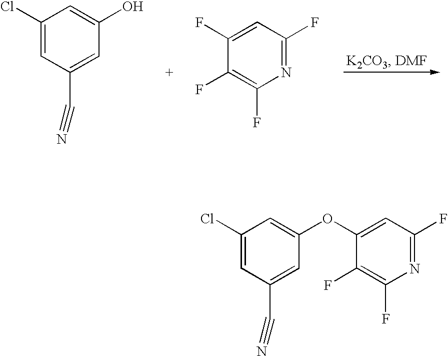

To 2,3,4,6-tetrafluoropyridine (5.9 g, 39 mmol) in DMF (20 mL) at r.t, 3-chloro-5-cyanophenol (6.0 g, 20 mmol) was added, followed by potassium carbonate (7.0 g, 51 mmol). Stirred for 1 h. Partitioned between ethyl acetate and water, organic layer washed with brine, water, dried over sodium sulfate, concentrated. Purified by flash column, eluted with 0% to 10% ethyl acetate in Hexane, obtained white solid 6.7 g, yield 61%.

### Step 2

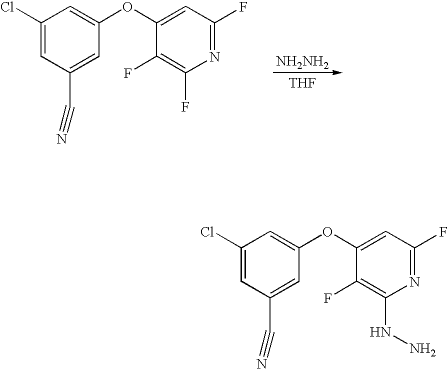

To 3-chloro-5-(2,3,6-trifluoro pyridin-4-yloxy)benzonitrile (6.7 g, 23.6 mmol) in 24 mL of THF, at r.t, hydrazine (1.5 mL, 47.3 mmol) was added. White precipitate appeared after 1 h. Removed solvent, the white residue triturated with hexane, obtained white solid 7.0 g, yield 100%.

### Step 3

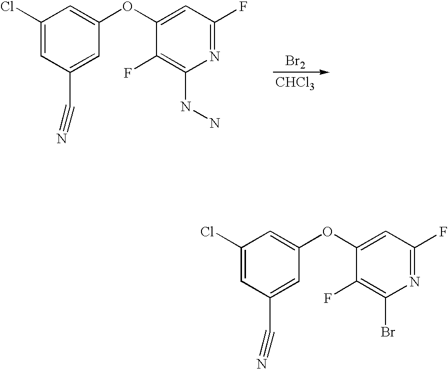

To 3-chloro-5-(3,6-difluoro-2-hydrazino-pyridin-4-yloxy)-benzonotrile (7.0 g, 23.6 mmol) suspended in 50 mL of Chloroform, was added bromine (2.44 mL, 47.2 mmol) dropwise. Reaction mixture became orange suspension. Heated to 60° C. for 6 h, then stood at r.t overnight. Diluted the reaction with DCM, washed organic layer with saturated sodium sulfite, water, dried over sodium sulfate, concentrated. Purified by flash column, eluted with 0% to 10% ethyl acetate in Hexane, obtained white solid 3.1 g, yield 38%.

### Step 4

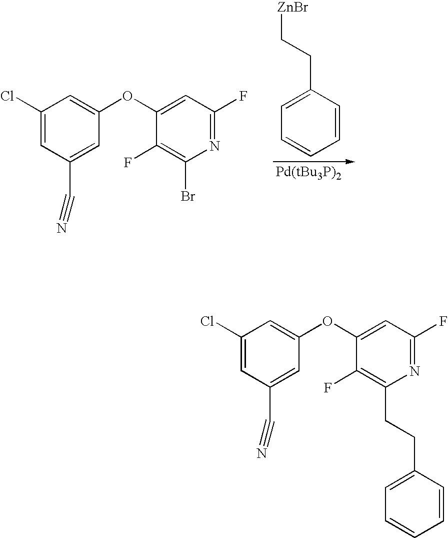

To 3-(2-bromo-3,6-difluoro-pyridin-4-yloxy)-5-chloro benzonitrile (1.5 g, 4.35 mmol) in 40 mL of dioxane, was added bis-(tri-tertbutyl phosphine)palladium(0), followed by 0.5 M phenethyzinc bromide (13 mL, 6.5 mmol) in THF. After stirring at r.t for 2 h, partitioned between ethyl acetate and brine, organic layer dried over sodium sulfate, concentrated. Purified by flash column, eluted with 0% to 10% ethyl acetate in Hexane, obtained colorless oil 1.1 g, yield 68%.

### Step 5

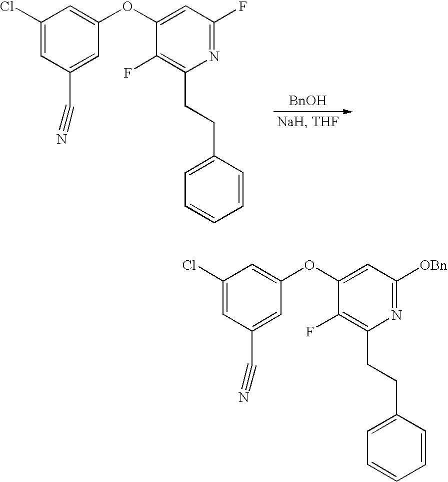

Sodium hydride (178 mg, 4.45 mmol) suspended in THF (20 ml) at r.t, benzyl alcohol (460 μL, 4.45 mmol) was added. After stirring for 10 minutes, a THF (20 mL) solution of 3-chloro-5-(3,6-difluoro-2-phenethyl-pyridin-4-yloxy)-benzonotrile (1.1 g, 4.45 mmol) was added. After 1 h, quenched the reaction with saturated aqueous ammonium chloride, extracted with ethyl acetate, dried over sodium sulfate, concentrated. Purified by flash column, eluted with 0% to 10% ethyl acetate in Hexane, obtained colorless oil 0.71 g, yield 58%.

### Step 6

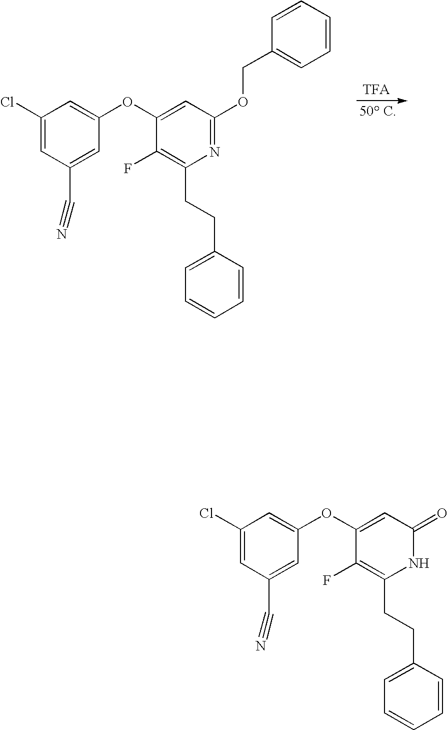

3-(6-Benzyloxy-3-fluoro-2-phenethyl-pyridin-4-yloxyl)-5-chloro-benzonitrile (0.71 g) in 20 mL of TFA, heated to 50° C. for 5 h. Removed most of TFA on Rotavap, washed with aqueous sodium bicarbonate, ethyl acetate extracted, dried over sodium sulfate, concentrated. Purified by flash column, eluted with 0% to 5% methanol in dichloromethane, obtained off white solid 0.26 g, yield 46%.

### Step 7

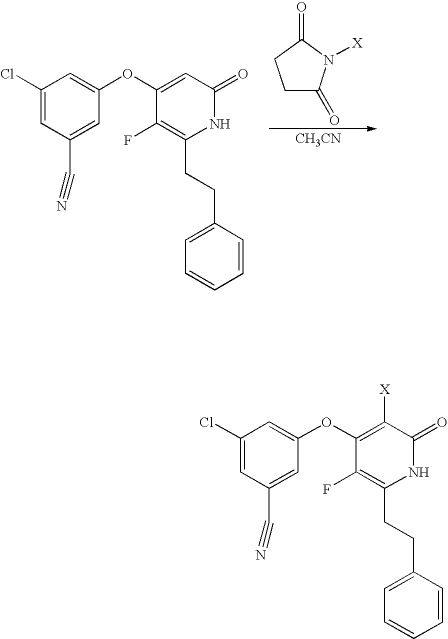

### X═Cl:

3-Chloro-5-(5-fluoro-2-oxo-6-phenethyl-1,2-dihydro-pyridin-4-yloxy)-benzonitrile (37 mg, 0.1 mmol) suspended in CH₃CN (1 mL) and isopropanol (1 mL) at

r.t, added N-chloro succinimide (13.4 mg, 0.1 mmol) and heated to 60° C. for 2 h. Removed solvent, purified by preparative HPLC to get product as white solid (8 mg), yield 20%.

### X═Br:

3-Chloro-5-(5-fluoro-2-oxo-6-phenethyl-1,2-dihydro-pyridin-4-yloxy)-benzonitrile (37 mg, 0.1 mmol) suspended in CH₃CN at r.t, added N-bromo succinimide (1.0 eq) and stirred for 1 h. Removed solvent, purified by preparative HPLC to get product (20 mg) as white solid, yield 46%.

### X═I:

3-Chloro-5-(5-fluoro-2-oxo-6-phenethyl-1,2-dihydro-pyridin-4-yloxy)-benzonitrile (176 mg, 0.476 mmol) suspended in ethyl acetate (10 mL) and acetic acid (1 mL), added N-iodo succinimide (107 mg, 1.0 eq) and heated to 50° C. for 4 h. Removed solvent, purified by flash column, eluted with 0% to 5% methanol in dichloromethane, got product (100 mg) as light yellow solid, yield 43%.

The following compounds were prepared generally as above using intermediate 3-(2-Bromo-3,6-difluoro-pyridin-4-yloxy)-5-chloro-benzonitrile (shown below): 1-20 and 1-21.

### Intermediate:

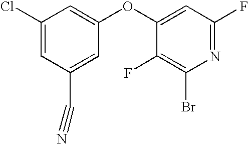

As described for compounds in the Sonagashira route above for analogous 5-H compounds followed by halogenation described in the previous example.

### Example 7

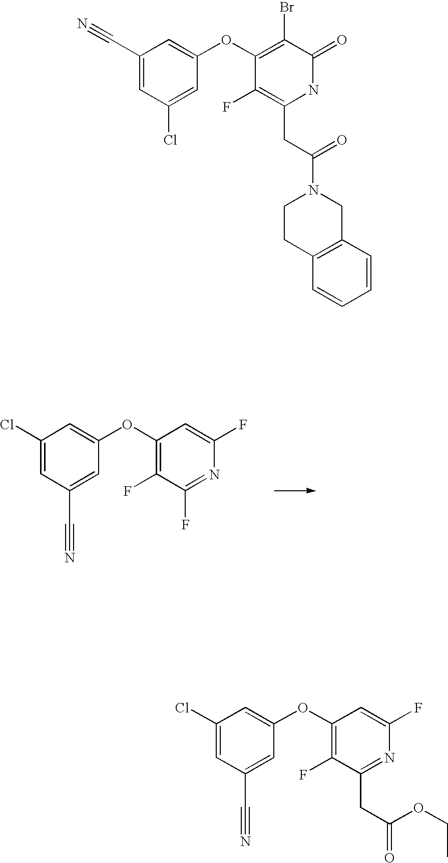

Ethyl-tert-butylmalonate (2.06 g, 1.05 equiv) in DMF (5 mL) was added to 60% NaH (1.05 g, 2.0 equiv) in DMF (25 mL) at 0° C., the entire mixture was then warmed to rt for 20 min, after which it was recooled and the pyridyl phenyl ether (3.75 g, 13.2 mmol) in DMF (5 mL) was slowly added. The reaction mixture was then allowed to slowly warm to rt over 2 hrs. The mixture was then recooled to 0° C., quenched with saturated NH4Cl, diluted with water, and then extracted with Et2O. The organic layers were washed with brine, dried over magnesium sulfate, and concentrated in vacuo. The residue obtained was redissolved in DCM (25 mL) and treated with TFA (10 mL) for 3 h. After which, the mixture was concentrated in vacuo, and chromatographed (SiO2, 5% to 15% EtOAc/hexanes) to provide ester product (2.7 g, 58%).

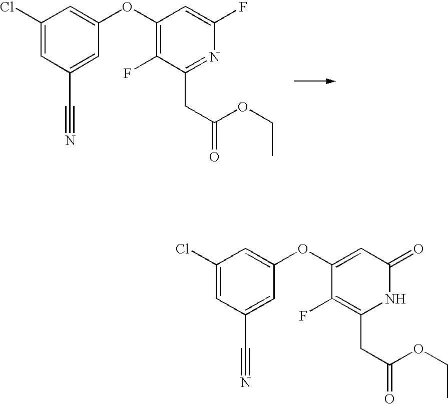

NaOAc (1.74 g, 3 equiv) was added to the ester (1.50 g, 4.26 mmol) in AcOH (20 mL). The mixture was then heated to 115° C. for 3 days, after which it was cooled, concentrated in vacuo, and chromatographed directly (SiO2, 1% to 10% MeOH/DCM) to provide pyridone product (580 mg, 39%).

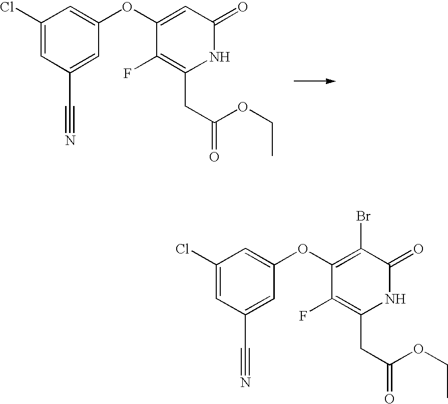

NBS (360 mg, 3.5 equiv) was added to a solution of the pyridone (200 mg, 0.57 mmol) in acetonitrile (3.5 mL). The mixture was then allowed to stir at rt for 2 h, upon which the mixture was quenched with saturated 1 M sodium bisulfite, diluted with water, and then extracted with EtOAc. The organic layers were washed with brine, dried over magnesium sulfate, and concentrated in vacuo. The residue obtained was redissolved in MeOH (5 mL) and treated with 1 M sodium bisulfite (5 mL) at 45° C. for 3 h. After which, the mixture was diluted with water, and extracted with EtOAc. The organic layers were washed with brine, dried over magnesium sulfate, concentrated in vacuo, and chromatographed (SiO₂, 1% to 10% MeOH/DCM) to provide bromo pyridone product (61 mg, 25%).

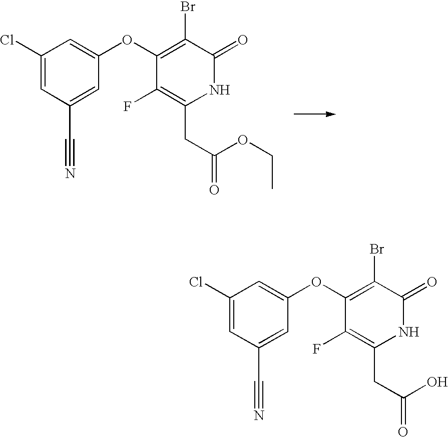

LiOH.H2O (18 mg, 2.25 equiv) in H2O (250 μL) was slowly added to a solution of the ester (80 mg, 0.19 mmol) in THF (1 mL) at 0° C. After 7 h 5% HCl was added, and the mixture extracted with EtOAc. The organic layers were washed with brine, dried over magnesium sulfate, and concentrated in vacuo. Trituration of the solid with ether and hexanes provided the acid (40 mg, 54%).

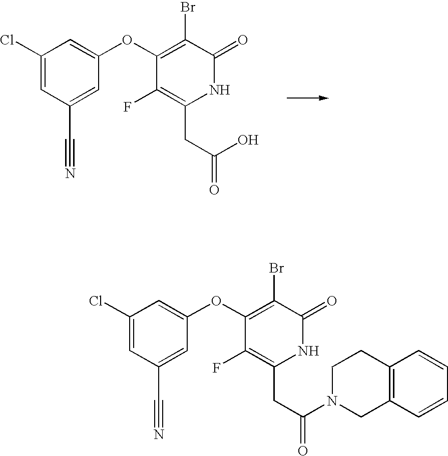

EDCI (16 mg, 1.7 equiv) was added to a solution of 1,2,3,4-tetrahydroisoquinoline (9 mg, 1.3 equiv), N-methylmorpholine (6 mg, 1.2 equiv), HOBT (7 mg, 1.05 equiv), DMAP (catalytic amount), and the phenyl acetic acid (20 mg, 0.050 mmol) in DMF (250 μL) at rt. This mixture was then stirred 4 h at rt, after NH4Cl was added, and the mixture extracted with CH2Cl2. The organic layers were washed with brine, dried over magnesium sulfate, and concentrated in vacuo. Preparative TLC (SiO2, 5% MeOH/CH2Cl2) provided the desired product (10 mg, 39%).

### Example 8

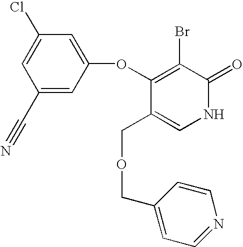

This example illustrates the synthesis of 3-[3-bromo-2-oxo-5-(pyridin-4-ylmethoxymethyl)-1,2-dihydro-pyridin-4-yloxy]-5-chloro-benzonitrile.

### Step 1. Preparation 4-Hydroxy-5-nitro-6-oxo-1,6-dihydro-pyridine-3-carboxylic acid ethyl ester

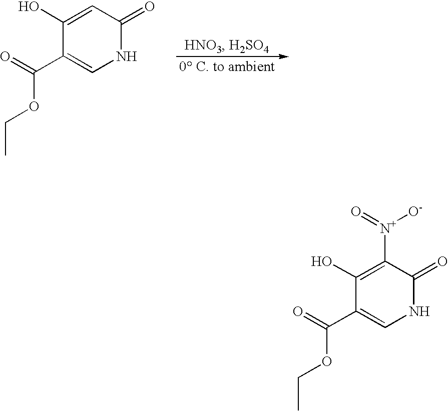

To a cold (ice bath) solution of 4-hydroxy-6-oxo-1,6-dihydro-pyridine-3-carboxylic acid ethyl ester (9.1 g, 49.7 mmol) in concentrated sulfuric acid (75 mL) was added nitric acid (2.9 mL, 64.6 mmol) via dropwise addition. The mixture was stirred for 1 hour and then the cooling bath was removed. After 15 minutes the mixture was poured into a beaker containing a 500 mL volume of ice. The material was stirred for 10 minutes and the precipitated product was collected by filtration. The precipitate was washed well with greater than 1 liter of water. The solid was dried in a vacuum oven providing the desired product as a light yellow white solid (6.8 g).

### Step 2. Preparation of 4-Chloro-5-nitro-6-oxo-1,6-dihydro-pyridine-3-carboxylic acid ethyl ester

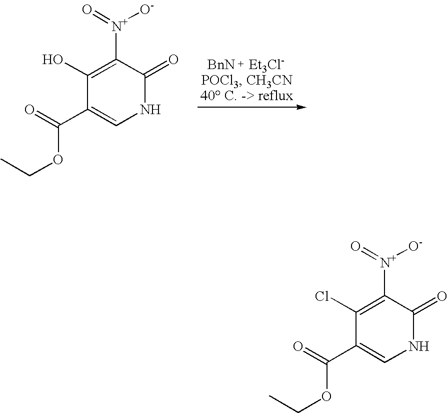

To a mixture of 4-hydroxy-5-nitro-6-oxo-1,6-dihydro-pyridine-3-carboxylic acid ethyl ester (6.8 g, 29.8 mmol) and benzyltriethylammonium chloride (27.15 g, 119 mmol) in dry acetonitrile (115 mL) was added phosphoryl chloride (12 mL, 131 mmol) via drop-wise addition. The material was heated to 40° C. (oil bath) for 30 minutes and then heated to reflux for 1 hour. The mixture was cooled to ambient. The solvent and volatiles were removed on the rotary evaporator. Water (115 mL) was added and the mixture was stirred for about 3 hours. The precipitated product is collected by filtration. The solid is washed well with water and dried in the vacuum oven, providing an off-white crystalline product (6.21 g).

Step 3. Preparation of 4-Chloro-6-methoxy-5-nitro-nicotinic acid ethyl ester

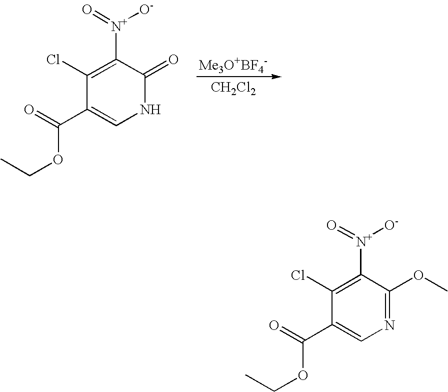

To a solution of 4-Chloro-5-nitro-6-oxo-1,6-dihydro-pyridine-3-carboxylic acid ethyl ester (7.5 g, 30.4 mmol) in dry dichloromethane (100 ml) was added trimethyloxonium tetrafluoroborate (4.59 g, 30.4 mmol) and the mixture was heated to reflux overnight. Additional trimethyloxonium tetrafluoroborate (2.3 g, 15.2 mmol) was added and heating was continued for 4 hours. The solution was cooled to ambient and water (50 ml) was added with stirring. Agitate and collect the CH₂Cl₂ phase. Back extract with CH₂Cl₂ (1×25 ml), combine the organic phases and dry over magnesium sulfate. Chromatography (SiO_{2 [}80 g], 20% EtOAc/Hexanes) gave the title compound as a white crystalline solid (6 g).

### Step 4 Preparation 4-(3-Bromo-5-chloro-phenoxy)-6-methoxy-5-nitro-nicotinic acid ethyl ester

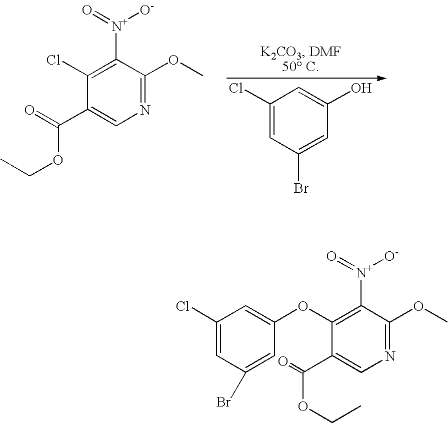

To a solution of 4-chloro-6-methoxy-5-nitro-nicotinic acid ethyl esterpyridine (1.61 g, 6.18 mmol) in dry DMF (14 mL) was added powdered potassium carbonate (1.71 g, 13.6 mmol) followed by 3-bromo-5-chloro-phenol (1.41 g, 6.8 mmol). The mixture was heated to 50° C. for 6 hours and then at 80° C. for 5 hours. Additional 3-bromo-5-chloro-phenol (220 mg) was added as well as K₂CO₃ (270 mg) and heating at 80° C. was continued for 4 hours. The material was cooled to ambient and concentrated (rotary evaporator/high vacuum pump). The remainder was taken up in ethyl acetate (60 ml) and water (60 ml). Agitate and collect the EtOAc phase. Back extract with EtOAc (2×40 ml), combine the organic phases and dry over magnesium sulfate. Chromatography (SiO_{2 [}60 g], 2-14% EtOAc/Hexanes) gave the title compound as a light yellow-brown solid (1.92 g).

### Step 5 Preparation of s-Amino-4-(3-bromo-5-chloro-phenoxy)-6-methoxy-nicotinic acid ethyl ester

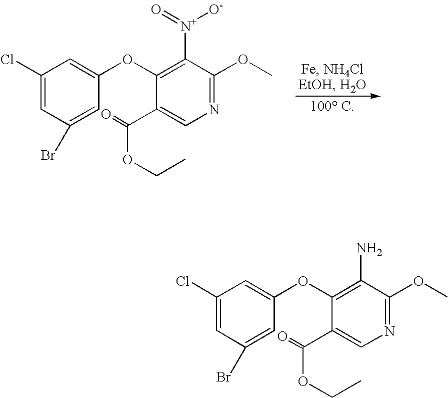

To a solution of 4-(3-bromo-5-chloro-phenoxy)-6-methoxy-5-nitro-nicotinic acid ethyl ester (1.91 g, 4.43 mmol) in ethanol (10 mL) and water (6 ml) was added electrolytic iron (1 g, 17.7 mmol) and ammonium chloride (957 mg, 17.7 mmol). The mixture was heated to 100° C. for 4 hours. The material was filtered (hot) through a plug of celite. Rinse well with hot EtOAc (about 100 ml). The filtrite was washed with an equal volume of brine and back extracted with EtOAc (2×50 ml). The title compound was obtained as a light yellow-brown solid (1.54 g).

### Step 6 Preparation of [5-amino-4-(3-bromo-5-chloro-phenoxy)-6-methoxy-pyridin-3-yl]-methanol

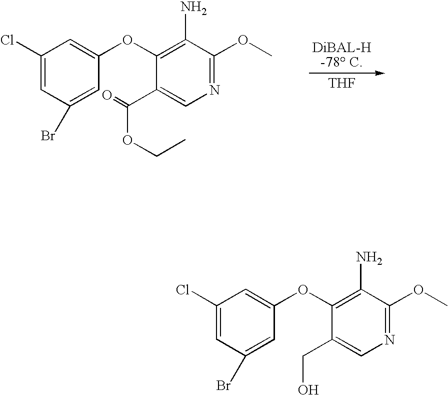

A solution of 5-Amino-4-(3-bromo-5-chloro-phenoxy)-6-methoxy-nicotinic acid ethyl ester (1.53 g, 3.82 mmol) in dry THF (45 mL) was cooled to −78° C. (acetone/dry ice bath) under a N₂ atmosphere. A solution of diisobutylaluminum hydride in CH₂Cl₂ (15 ml, 1.4 M) was added via drop-wise addition. The mixture was stirred for 5 minutes and then warmed to 0° C. An aqueous solution of 10% Rochelle's salt (75 ml) was added and the mixture was stirred for 1.5 hours. The material was transferred to a separatory funnel and water (40 ml) was added with EtOAc (about 100 ml). The material was agitated and the EtOAc phase collected and washed with brine (100 ml). The aqueous phase was back extracted with EtOAc (2×75 ml). The combined organic phases were dried (MgSO4), filtered and concentrated on the rotovap. Purification by preparative TLC (50% EtOAc/Hexanes) provided the final product as a light yellow-brown solid (900 mg).

### Step 7 Preparation of 3-(3-amino-5-hydroxymethyl-2-methoxy-pyridin-4-yloxy)-5-chloro-benzonitrile

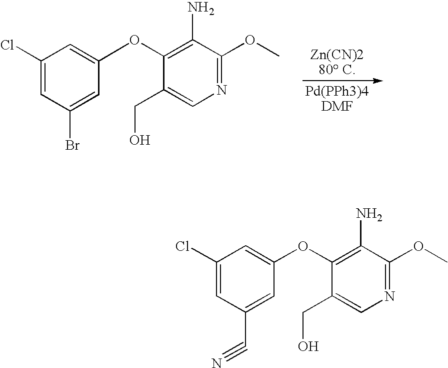

A mixture of [5-amino-4-(3-bromo-5-chloro-phenoxy)-6-methoxy-pyridin-3-yl]-methanol (847 mg, 2.36 mmol), Zn(CN)₂ (277 mg, 2.36 mmol) and Pd(PPh3)₄ (278 mg, 0.24 mmol) in dry DMF (20 mL) was de-gassed (5 vacuum/argon cycles). The material was heated to 80° C. for 8 hours under argon balloon. The material was cooled to ambient and concentrated (rotary evaporator/high vacuum pump). The remainder was taken up in ethyl acetate (50 ml) and water (50 ml). Agitate and collect the EtOAc phase and wash with brine (50 ml). Back extract with EtOAc (2×40 ml), combine the organic phases and dry over magnesium sulfate. Chromatography (SiO_{2 [}40 g], 20-60% EtOAc/Hexanes) gave the title compound as a off-white solid (648 mg).

### Step 8 Preparation of 3-(3-bromo-5-hydroxymethyl-2-methoxy-pyridin-4-yloxy)-5-chloro-benzonitrile

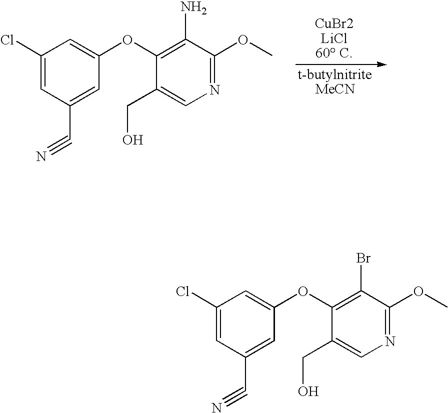

A mixture of Cu(II) Br₂ (366 mg, 1.64 mmol) and LiBr (357 mg, 4.11 mmol) in dry acetonitrile (5 mL) was heated to 60° C. Tert-butylnitrite (0.31 ml, 2.4 mmol) was added dropwise and the material stirred for 25 minutes. A solution of 3-(3-amino-5-hydroxymethyl-2-methoxy-pyridin-4-yloxy)-5-chloro-benzonitrile (418 mg, 1.37 mmol) in acetonitrile (4 ml) was added dropwise and the mixture was stirred at 60° C. for 3 hours. The material was cooled to ambient and poured into a 2 phase mixture of 10% aqueous HBr (35 ml) and ethyl acetate (40 ml). Agitate and collect the EtOAc phase and wash consecutively with equal volumes of water and brine. Back extract with EtOAc (2×40 ml), combine the organic phases and dry over magnesium sulfate. Chromatography (preparative TLC, 47% EtOAc/Hexanes) gave the title compound as a yellow-brown solid (259 mg).

### Step 9 Preparation of 3-(3-bromo-5-bromomethyl-2-methoxy-pyridin-4-yloxy)-5-chloro-benzonitrile

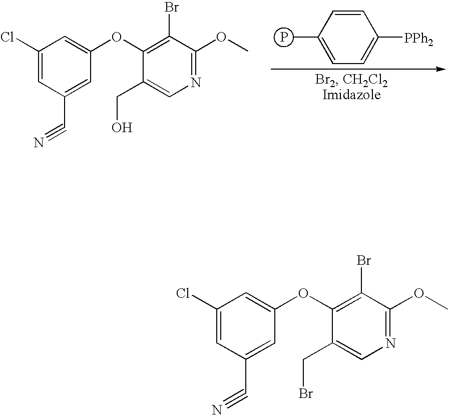

A oven dried flask is charged with bromine (68 mg, 0.42 mmol) and taken up in CH₂Cl₂ (4 ml). Imidazole (29 mg, 0.42 mmol) and 4-diphenylphosphino polystyrene resin (140 mg, 3 mmol/g) are added and the mixture was stirred for 5 minutes. A solution of 3-(3-bromo-5-hydroxymethyl-2-methoxy-pyridin-4-yloxy)-5-chloro-benzonitrile (118 mg, 0.38 mmol) in CH₂Cl₂ (2 ml) was added dropwise. The mixture was stirred for 15 minutes and then filtered through a plug of celite. The celite plug was rinsed well with wet CH₂Cl₂ (40 ml). The CH₂Cl₂ filtrate was transferred to a separatory funnel and washed consecutively with equal volumes of 5% aqueous sodium thiosulfate and then brine. Back extract with CH₂Cl₂ (2×40 ml), dry (MgSO4), filter and strip the solvent to obtain the title compound as a light yellow oil (131 mg).

### Step 10 Preparation of 3-[3-bromo-2-methoxy-5-(pyridin-4-ylmethoxymethyl)-pyridin-4-yloxy]-5-chloro-benzonitrile

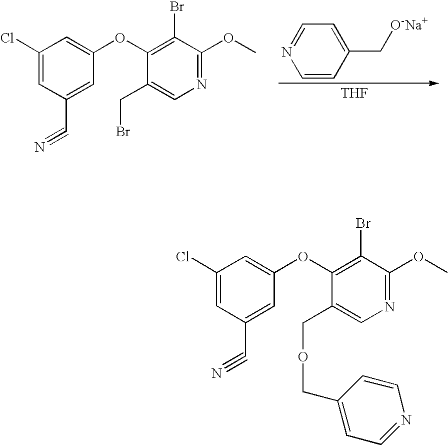

A solution of pyridine-4-methanol (73 mg, 0.67 mmol) in dry THF (3 mL) was cooled to 0° C. (ice bath) under a N₂ atmosphere. Powdered NaH (29 mg, 0.7 mmol, 60% in oil) was added and the mixture stirred for 10 minutes at which point the cooling bath is removed. After 20 minutes a solution of 3-(3-bromo-5-bromomethyl-2-methoxy-pyridin-4-yloxy)-5-chloro-benzonitrile (131 mg, 0.303 mmol) in dry THF (2.5 ml) is added via drop-wise addition. The mixture was stirred for 3 hours and then quenched with a saturated solution of aqueous NH₄Cl (5 ml), water (30 ml) and EtOAc (30 ml). The material was shaken in a separatory funnel and the EtOAc phase collected and washed with brine (30 ml). Back extract with EtOAc (2×30 ml), dry (MgSO4), filter and strip. Chromatography (preparative TLC, 4% MeOH/CH₂Cl₂) gave the title compound as a light yellow semi-solid (34 mg).

### Step 11 Preparation of 3-[3-bromo-2-oxo-5-(pyridin-4-ylmethoxymethyl)-1,2-dihydro-pyridin-4-yloxy]-5-chloro-benzonitrile

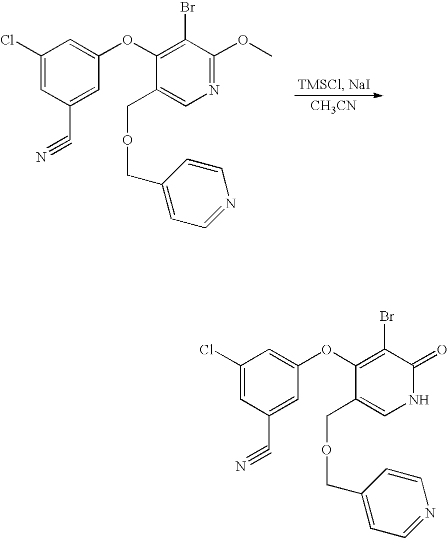

An oven dried flask is charged with 3-[3-bromo-2-methoxy-5-(pyridin-4-ylmethoxymethyl)-pyridin-4-yloxy]-5-chloro-benzonitrile (34 mg, 0.074 mmol) and taken up in CH₃CN (5 ml). Sodium iodide (27 mg, 0.19 mmol) is added and the mixture was cooled to 0° C. (ice bath) under N₂ atmosphere. TMSCl (0.02 ml, 0.19 mmol) was added drop-wise and the mixture was stirred for 5 minutes at which point the cooling bath is removed. The mixture was stirred for 2.5 hours and then treated consecutively with 1 NHCl (0.25 ml), 5% aqueous NaHSO₃ (0.5 ml). Stir vigorously for 2 minutes and then add brine (10 ml), water (10 ml) and then EtOAc (30 ml). Transfer to a separatory funnel, shake and isolate the organic phase. Wash with brine (25 ml) and back extract with EtOAc (2×25 ml). Combine the organics, dry (MgSO4), filter and strip. Chromatography (preparative TLC, 5% MeOH/CH₂Cl₂) gave the title compound as a light yellow powder (7 mg).

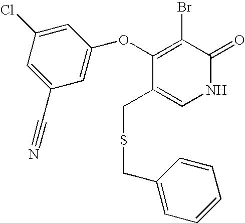

This example illustrates the synthesis of 3-(5-benzylsulfanylmethyl-3-bromo-2-oxo-1,2-dihydro-pyridin-4-yloxy)-5-chloro-benzonitrile.

The preparation of this material is analogous to that shown in steps 10 and 11, with modifications shown below:

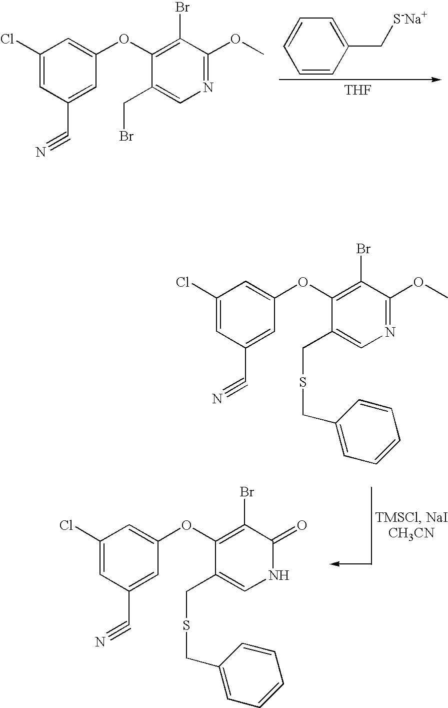

### Example 9

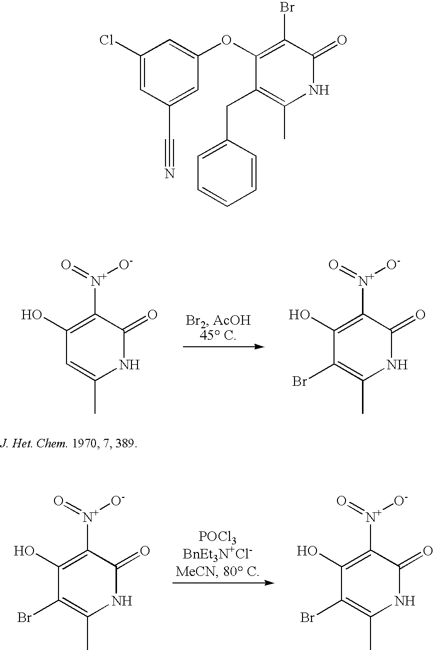

POCl₃ (11.2 mL, 4.4 equiv) was added to a mixture of benzyltriethylammonium chloride (25.5 g, 4.0 equiv) and 3-nitro-4-hydroxy-5-bromo-2-pyridone (14.0 g, 56.0 mmol) in acetonitrile (100 mL). This mixture was stirred at 40° C. for 30 min, after which it was refluxed for 1 h. Upon cooling, the mixture was concentrated in vacuo to remove excess reagents, and then 100 mL of H₂O was added at 0° C. After stirring overnight, 3-nitro-4-chloro-5-bromo-2-pyridone was obtained (13.9 g) as cream colored solid.

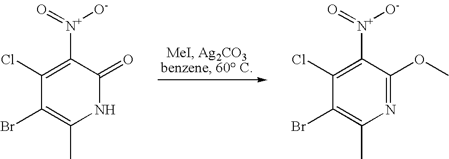

Silver carbonate (5.25 g, 0.51 equiv) and methyl iodide (2.56 mL, 1.05 equiv) were slowly added to a solution of bromopyridone (10.0 g, 37.4 mmol) in benzene (125 mL). After heating for 18 hrs at 60° C. in a sealed tube, the reaction mixture was cooled to rt, filtered over celite, washed with EtOAc, and concentrated in vacuo. The resulting material was chromatographed directly (SiO₂, 3% to 15% EtOAc/hexanes) to provide methoxypyridine product (6.2 g).

60% NaH (1.2 g, 1.3 equiv) was added to a solution of 3-chloro-5-cyanophenol (5.12 g, 1.4 equiv) in DMF (80 mL) at 0° C. This solution was then stirred at room temperature until all of the NaH had reacted (˜30 min). After recooling to 0° C., methoxypyridine (6.7 g, 23.8 mmol) was added and the purple colored solution was allowed to stir from 0° C. to rt. After 1 h the mixture was quenched with sat. NH₄Cl, extracted with EtOAc, washed with water and brine, dried over MgSO₄, and concentrated in vacuo. This material was chromatographed (SiO₂, 15% to 33% EtOAc/hexanes) to provide slightly impure coupled product (˜5 g) and recovered methoxypyridine starting material (˜1 g).

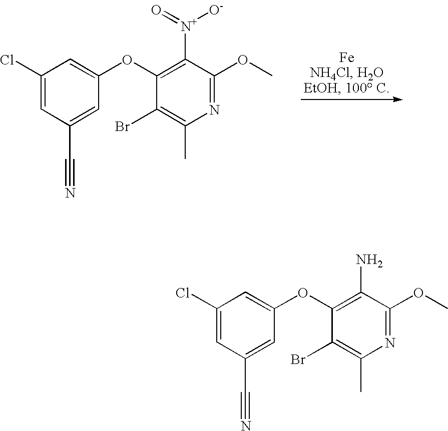

To a solution of coupled methoxypyridine (5.0 g, 12.5 mmol) in EtOH (60 mL) containing ammonium chloride (2.68 g, 4.0 equiv) and H₂O (20 mL) was added electrolytic Fe powder (2.79, 4.0 equiv) with rapid stirring at 50° C. The temperature of the reaction was then raised to 100° C. After 1 hr the reaction was deemed complete by TLC. While still hot, celite and EtOAc were added and the entire mixture was filtered over an additional portion celite. Concentration in vacuo gave a residue (˜4.6 g) that was somewhat insoluble and difficult to purify by chromatography. This material should be recrystallized or carried on crude.

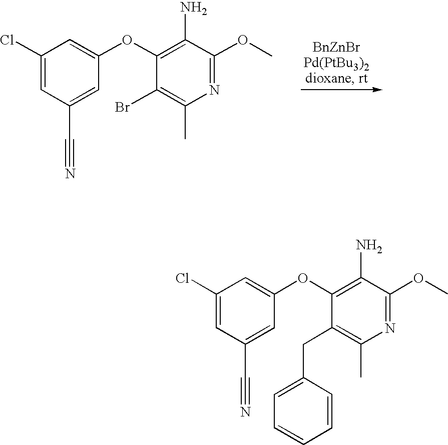

Benzylzinc bromide (3.26 mL, 0.5M, 1.2 equiv) was added to a solution of bis(tritertbutylphosphine) palladium (104 mg, 0.15 equiv) and bromo aniline (500 mg, 1.36 mmol) from the previous step in dioxane (9 mL) at rt. This solution was then stirred at room temperature until deemed complete by LC/MS (˜2 h). Upon quenching with sat. NH₄Cl, the mixture was extracted with EtOAc, washed with water and brine, dried over MgSO₄, concentrated in vacuo, and chromatographed (SiO₂, 10% to 33% EtOAc/hexanes) to provide coupled product (260 mg, 51%).

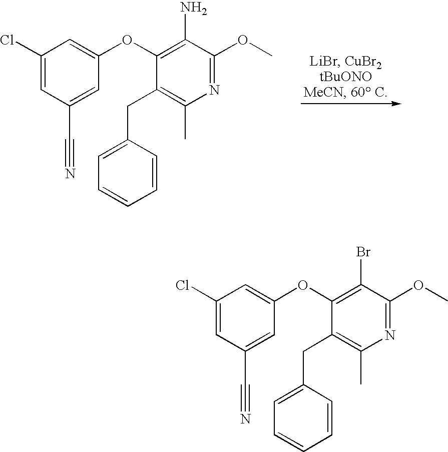

tBuONO (143 μL, 1.75 equiv) was slowly added to a solution of CuBr (184 mg, 1.2 equiv) and LiBr (179 mg, 3 equiv) in acetonitrile (3.5 mL) at 60° C. To this was then added the product from the previous step (260 mg, 0.69 mmol) in acetonitrile (3.5 mL) dropwise. After heating for 1 h at 60° C. the reaction mixture was cooled, and quenched with 5% aq. HBr. The mixture was extracted with EtOAc, washed with water and brine, dried over MgSO₄, concentrated in vacuo, and chromatographed (SiO₂, 10% to 33% EtOAc/hexanes) to provide bromide (40 mg, 46%).

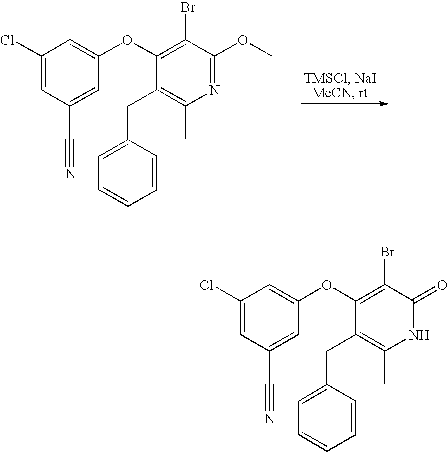

TMSCl (87 μL, 2.5 equiv) was slowly added to a solution of NaI (103 mg, 2.5 equiv) and bromide (122 mg, 0.28 mmol) in acetonitrile (2 mL). After stirring for 3 h at rt the reaction mixture was quenched with aq. sodium thiosulfate. The resulting mixture was extracted with EtOAc, washed with water and brine, dried over MgSO₄, concentrated in vacuo, and chromatographed (SiO₂, 1% to 10% MeOH/DCM) to provide pyridone (50 mg, 42%).

The following compounds were prepared using the above methods: II-11 and II-6.

### Step 1

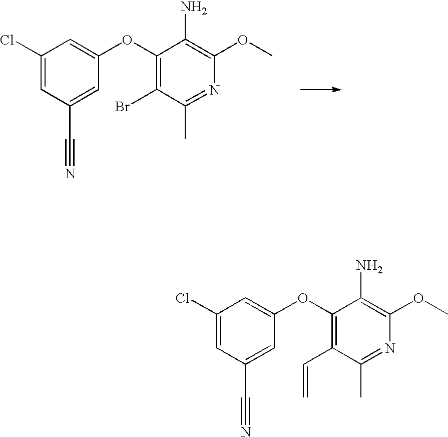

Vinyltributyltin (420 mg, 0.95 equiv) was added to a solution Pd(PPh3)4 (244 mg, 0.15 equiv) and bromopyridine (520 mg, 1.41 mmol) in DMF (7 mL) and the mixture was heated to 100° C. After 7 h, the mixture was cooled, extracted with ether, washed with water and brine, dried over MgSO₄, and concentrated in vacuo. The resulting mass was chromatographed (SiO₂, 10% to 33% EtOAc/hexanes) to provide vinylated product (150 mg, 34%).

### Step 2

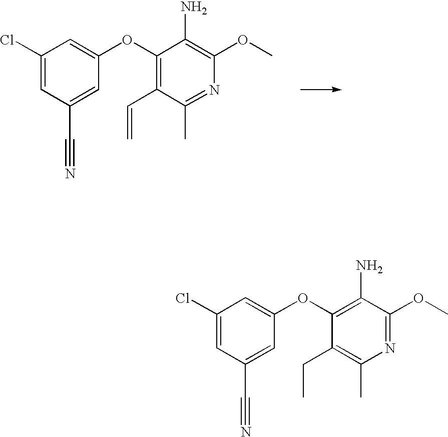

10% Pd/C (14 mg) was added to a solution of vinyl pyridine (140 mg, 0.44 mmol) in EtoAc (4 mL), MeOH (2 mL), and DMF (0.4 mL). The mixture was then stirred under an atmosphere of H₂ for 5 h. After which, the mixture was filtered over celite and concentrated in vacuo. Analysis of the crude mass by LC/MS revealed that the material was 80% pure. This material was carried on to the next step without any further purification.

### Step 3

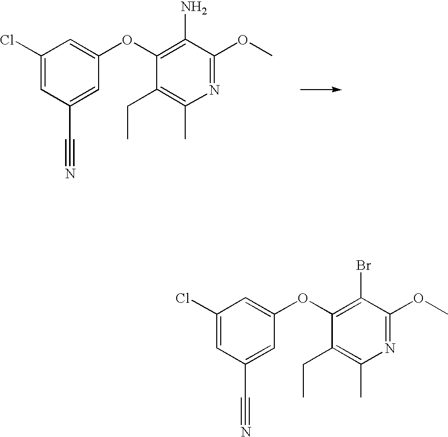

Carried out as described in the above example above

### Step 4

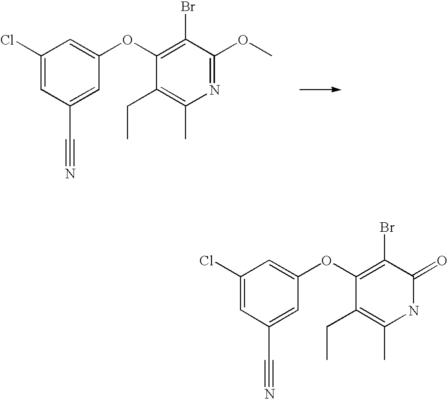

Carried out as described in the above example above

### Example 10

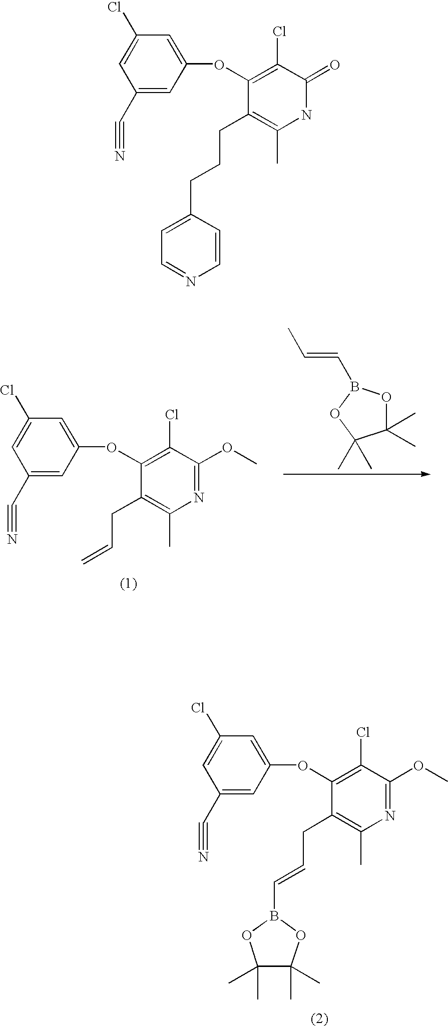

In a 50 ml Schlenck flask was placed 1.78 g (5.09 mmole) of (1), 0.216 g (0.25 mmole, 5%) of Grubb's catalyst (second generation), 1.71 g (10.2 mmole, 2 equivalent) of propenyl boronic acid pinacol ester, and 32 ml of dichloromethane. The mixture was evacuated and back filled with argon and refluxed at 50° C. for 8 hrs. The solvent was evaporated and the residue was purified by flash chromatography on silica gel eluting with 0 to 10% ethyl acetate/hexane. Obtained 1.44 g of (2), 59% yield as a light brown solid.

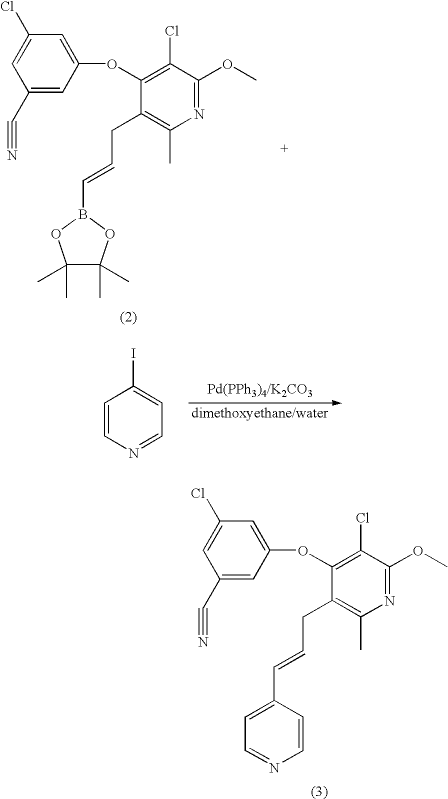

In a 25 ml Schlenck flask was placed 0.200 g (0.42 mmole) of (2) in 4 ml of 1,2-dimethoxyethane, 0.104 g of 4-iodopyridine, 24 mg (0.021 mmole) of tetrakis(triphenylphosphine)palladium, and 0.145 g (1.05 mmole) of potassium carbonate in 0.5 ml of water. The mixture was stirred, evacuated, and back filled with argon. The mixture was stirred and heated at 90° C. for 18 hrs. The solvent was evaporated, triturated the residue with 30 ml of 50% ethyl acetate/dichloromethane, and the crude product was purified by flash chromatography on silica gel eluting with 0 to 25% ethyl acetate/dichloromethane. Obtained 0.145 g of (3), 81% yield as a pink solid. Mass spec, (M+H)+=426.

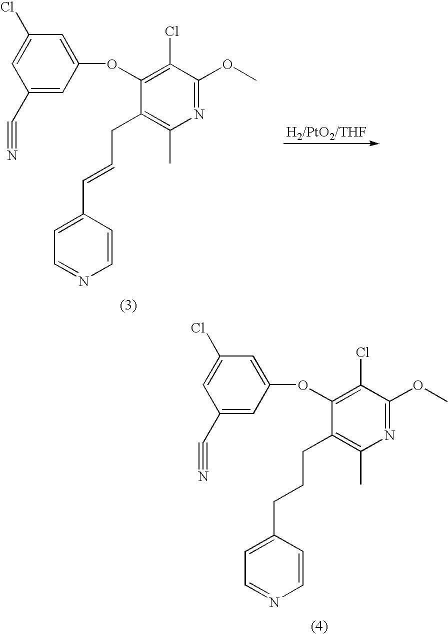

In a flask was placed 0.129 g (0.30 mmole) of (3) dissolved in 13 ml of THF and added 26 mg of platinum oxide. The mixture was stirred under a balloon of hydrogen gas for 12 hrs. Filtered through a pad of Celite, evaporated, and the residue was purified by chromatography on silica eluting with 0 to 25% ethyl acetate/dichloromethane. Obtained 81 mg of (4), 62% yield as a white solid. Mass spec, (M+H)+=428.

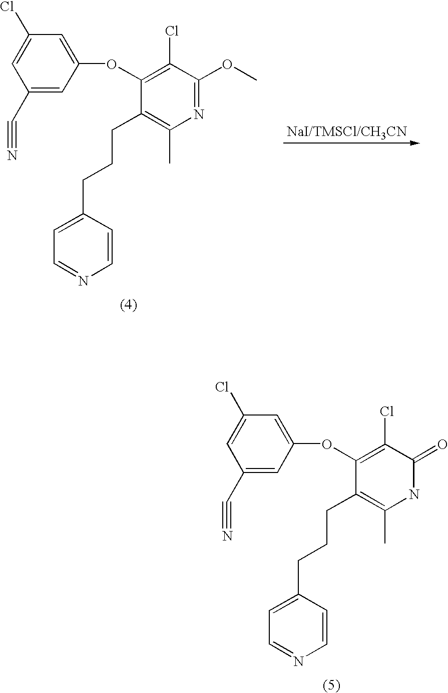

In a flask was placed 75 mg (0.175 mmole) of (4) dissolved in 4 ml of acetonitrile, added 66 mg (0.437 mmole) of sodium iodide, and then added 48 mg (0.437 mmole) of chorotrimethylsilane. The cloudy mixture was stirred at room temperature under a nitrogen atmosphere for 1 hr. The mixture was poured into 100 ml of ethyl acetate, washed with 10 ml of saturated sodium bicarbonate solution, 10 ml of 10% sodium bisulfite solution, 10 ml of brine, dried over magnesium sulfate, evaporated, and the residue purified by chromatography on silica gel eluting with 0 to 10% methanol/dichloromethane. Obtained 56 mg of (5), 77% yield as a white solid. Mass spec, (M+H)+=414.

The following compounds were prepared according the methods described above: II-17 and II-14.

### Example 11

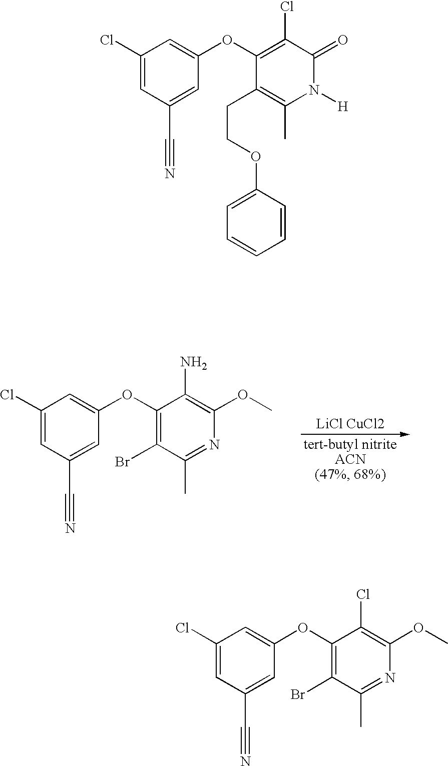

To a solution of copper chloride (0.878 g, 6.53 mmol) and lithium chloride (0.481 g, 11.4 mmol) in acetonitrile (20 mL) at 60° C. was added tert-butyl nitrite (1.31 mL, 9.94 mmol, 1.75 eq. 90%) dropwise. After 25 min. at 60° C., the pyridinyl compound was added. After 3 h. at room temperature, the reaction mixture was poured into saturated ammonium chloride (15 mL). Organic layer was washed with saturated ammonium chloride (15 mL), dried (MgSO₄), filtered and concentrated in vacuo to give a foamy, brown oily residue, which was flashed on silica (SiO₂, 5% EtOAc/Hexanes) to give the product as a white solid. (1.490 g, 68%)

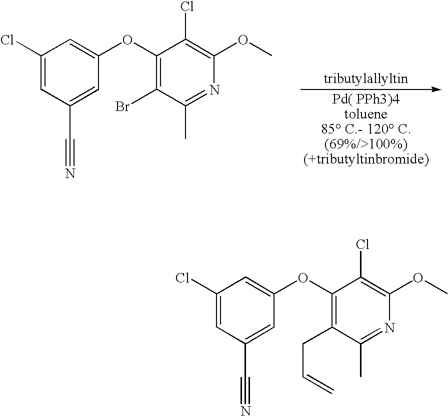

To a suspension of the pyridine (1.37 g, 3.53 mmol) and Pd(PPh₃)₄ (0.408 g, 0.353 mmol) in toluene (10 mL) was added tributyl allyl tin (1.23 mL, 3.88 mmol, 1.1 eq. 97%). The yellow reaction mixture was heated at 120° C. After 16 h., the reaction mixtured was cooled to room temperature and concentrated in vacuo to give a green oil, which was taken up in ether (20 mL) and washed with brine (20 mL), dried (MgSO4), filtered and concentrated in vacuo to give a yellow oil. Purification by flash chromatography (SiO2, 5% EtOAc/Hexanes) gave the product as a colorless oil (0.850 g, 69%).

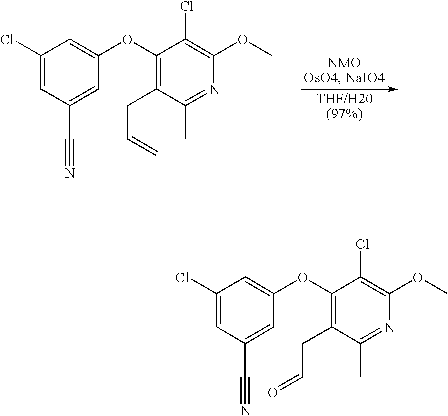

To a solution of the allylarene (0.814 g, 2.33 mmol) in THF (11 mL) and H₂O (2.4 mL) was added osmium tetraoxide (47 mg, 0.183 mmol), followed by N-methyl morpholine N-oxide (0.630 g, 4.58 g) at 0° C. The reaction mixture was warmed to room temperature over 1 h. and stirred for 12 h. To the brown reaction mixture was added 10% Na₂S₂O₃ in an aqueous solution (800 mg in 8 mL H₂O) and the reaction mixture was stirred. After 30 min., the reaction mixture was extracted with EtOAc(2×10 mL) and the combined organic extracts were dried over MgSO₄, filtered, and the volume of the solvent was reduced to 3 mL and filtered through a short plug of silica with EtOAc. The solvent was removed to give a foamy, white solid, which was taken up in MeOH (6 mL) and H₂O (6 mL) and stirred with sodium periodate (0.748 g, 1.5 eq.). After stirring the white slurry for 1.5 hr, the reaction mixture was filtered to rid of the salt and diluted with EtOAc, washed with brine (2*5 mL), dried (MgSO₄), and concentrated in vacuo to give the product as a white foamy solid (0.792 g, 97%).

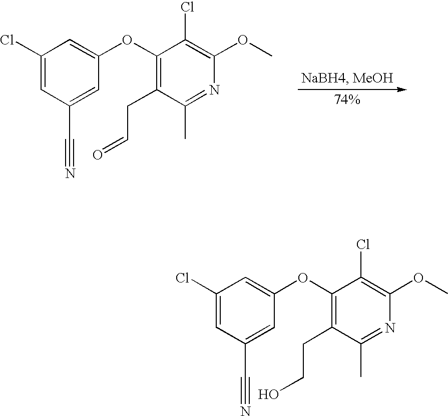

To a solution of aldehyde (0.792 g, 2.26 mmol) in methanol (16 mL) was added sodium borohydride (94 mg, 2.48 mmol) at 0° C. Bubbling occurred and the color turned pink orange and a solid started to precipitate out of solution with further stirring. After 20 min. at 0° C., the reaction mixture was partitioned between H₂O (15 mL) and EtOAc (15 mL). The organic layer was washed with brine (15 mL), dried (MgSO₄), filtered and concentrated in vacuo to give an orange solid, which was flashed (SiO₂, 40% EtOAc in Hexanes) to give the product as a white solid (0.591 g, 74%).

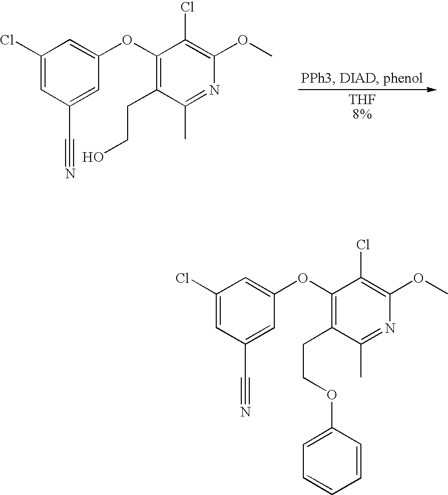

To a solution of the alcohol (0.250 g, 0.708 mmol) in THF (3 mL) was added PPh₃ (0.371 g, 1.42 mmol), phenol (0.067 g, 0.708 mmol) and diisopropylazodiacetate (0.151 mL, 0.779 mmol) dropwise at 0° C. The reaction mixture was gradually warmed to room temperature and stirred for 12 h. The solvent was removed under reduced pressure to give an oil, which was flashed (SiO₂, 0%-100% 60/10/1 CH₂Cl₂/MeOH/NH₄OH in CH₂Cl₂ to give the product (23 mg, 8%).

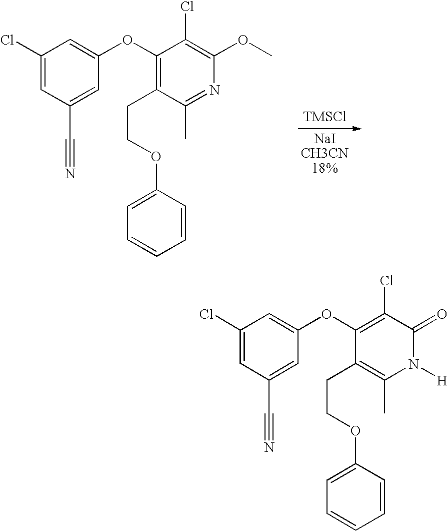

To a solution of the pyridinyl compound in acetonitrile (1 mL) was added sodium iodide (20 mg, 0.134 mmol), followed by a dropwise addition of trimethylsilylchloride (0.017 mL, 0.134 mmol) at 0° C. After 20 min., ice bath was removed and the reaction mixture was stirred at room temperature. After 3 h., the reaction mixture diluted with EtOAc (5 mL) and was poured into a mixture of saturated aqueous sodium bicarbonate (3 mL) and H₂O (3 mL). The organic layer was washed with brine (5 mL), dried (MgSO₄), filtered and concentrated in vacuo to give a yellow oil, which was flashed on preparative TLC (50% 60/10/1 CH₂Cl₂/MeOH/NH₄OH in CH₂Cl₂) to give an off-white solid (4 mg, 18%).

### Example 12

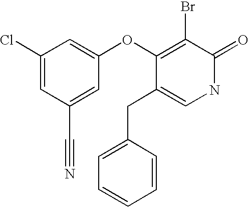

Prepared according to the literature:

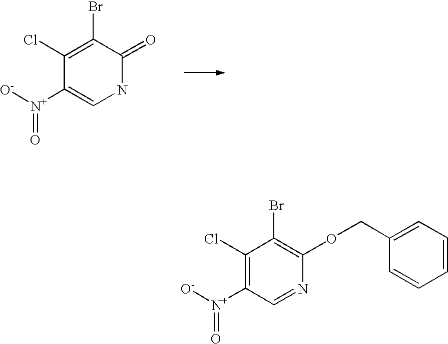

Silver carbonate (4.73 g, 0.51 equiv) and benzyl bromide (4.00 mL, 1.05 equiv) were slowly added to a solution of bromopyridone (8.5 g, 33.6 mmol) in benzene (120 mL). After heating for 18 hrs at 60° C., the reaction mixture was cooled to rt, filtered over celite, washed with EtOAc, and concentrated in vacuo. The resulting material was chromatographed directly (SiO₂, 3% to 15% EtOAc/hexanes) to provide benzyloxypyridine product (5.1 g, 44%).

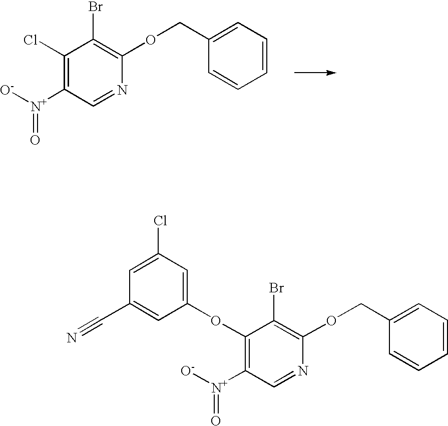

3-chloro-5-cyanophenol (2.23 g, 1.00 equiv) and potassium carbonate (3.50 g, 1.75 equiv) were added to a solution of nitro compound from the previous step (5.00 g, 14.55 mmol) in THF (50 mL). After heating for 20 hrs at 60° C. the reaction mixture was cooled to rt, and poured into water (200 mL). The mixture was extracted with ether, washed with water and brine, dried over MgSO₄, and concentrated in vacuo to give crude diaryl ether (˜7 g) which was carried on to the next step.

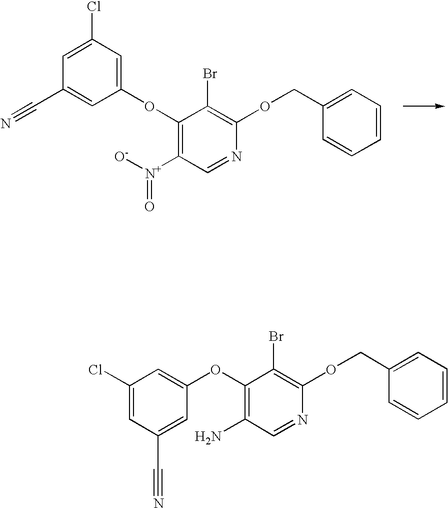

To a solution of diaryl ether (6.5 g, 14.1 mmol) in EtOH (50 mL) and EtOAc (10 mL) containing ammonium chloride (2.70 g, 3.5 equiv) and H₂O (15 mL) was added electrolytic Fe powder (2.60, 3.5 equiv) with rapid stirring at 50° C. The temperature of the reaction was then raised to 100° C. After 3 hr the reaction was deemed complete by TLC. While still hot, celite and EtOAc were added, and the entire mixture was filtered over an additional portion celite. Concentration in vacuo gave a residue (˜4.8 g) that was sufficiently pure to carry on to the next step.

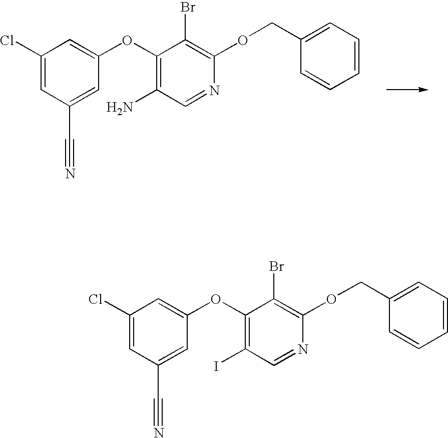

tBuONO (3.1 mL, 2.2 equiv) was slowly added to a suspension of the aniline (4.6 g, 10.68 mmol) in diiodomethane (17.3 mL, 20 equiv), and the mixture was then heated to 60° C. After 30 min, the mixture was cooled and chromatographed directly (SiO₂, 1% to 10% EtOAc/hexanes) to provide the iodide (2.75 g, 48%).

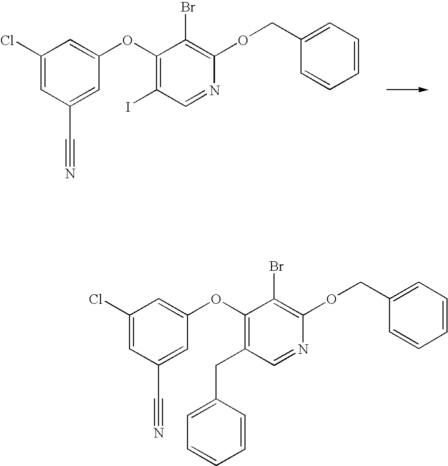

Benzylzinc bromide (450 μL, 0.5M, 1.2 equiv) was added to a solution of bis(tritertbutylphosphine) palladium (5 mg, 0.05 equiv) and the iodide (100 mg, 0.19 mmol) in dioxane (1 mL) at rt. This solution was then stirred at room temperature until deemed complete by LC/MS (˜4 h). Upon quenching with sat. NH₄Cl, the mixture was extracted with EtOAc, washed with water and brine, dried over MgSO₄, concentrated in vacuo, and chromatographed (SiO₂, 10% to 33% EtOAc/hexanes) to provide coupled product (46 mg, 50%).

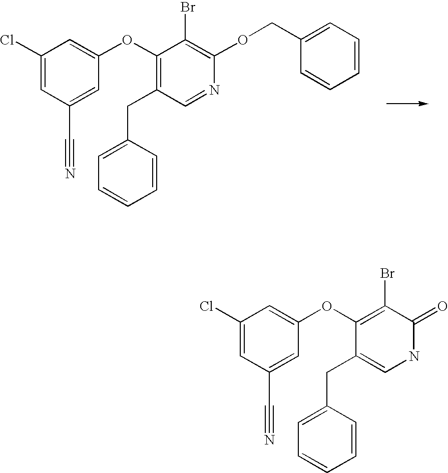

TFA (1 mL) was added to a solution ester (45 mg, 0.092 mmol) in DCM (2 mL) at rt. This solution was stirred at rt for 2 h, after which the mixture was concentrated in vacuo, and chromatographed (SiO₂, 2% to 10% MeOH/DCM) to provide pyridone product (18 mg, 47%).

The following compounds were prepared using the following allyl intermediate:

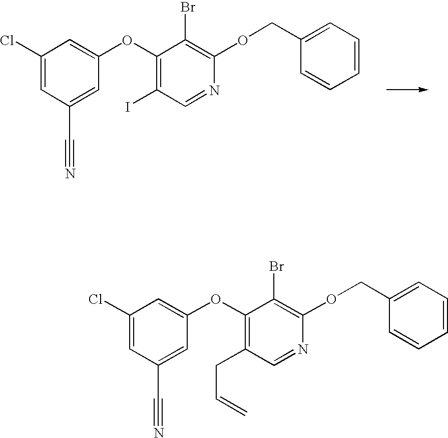

Allyltributyltin (1.36 g, 1.2 equiv) was added to a solution Pd(PPh3)4 (640 mg, 0.15 equiv) and the benzyloxypyridine (2.00 g, 3.69 mmol) in DMF (12.5 mL) and the mixture was heated to 100° C. After 4 h, the mixture was cooled, extracted with ether, washed with water and brine, dried over MgSO₄, and concentrated in vacuo. The resulting mass was chromatographed (SiO₂, 3% to 10% EtOAc/hexanes) to provide allylated product (1.4 g, 83%).

Using this intermediate the compounds were prepared using the oxidative cleavage/Mitsunobu route (and substituting the above TFA deprotection) described in the previous example: II-12, II-7, and II-9.

### Dosage and Administration

The compounds of the present invention may be formulated in a wide variety of oral administration dosage forms and carriers. Oral administration can be in the form of tablets, coated tablets, dragees, hard and soft gelatine capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The term “excipient” as used herein includes both one and more than one such excipient.

The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. N-acylsulfonamides have an acidic proton which can be abstracted to form a salt with an organic or inorganic cation.

The preferred pharmaceutically acceptable salts are the salts formed from acetic acid, hydrochloric acid, sulphuric acid, methanesulfonic acid, maleic acid, phosphoric acid, tartaric acid, citric acid, sodium, potassium, calcium, zinc, and magnesium. It should be understood that all references to pharmaceutically acceptable salts include solvent addition forms (solvates) or crystal forms (polymorphs) as defined herein, of the same acid addition salt.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to a skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 100 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

In embodiments of the invention, the active compound or a salt can be administered in combination with another antiviral agent, such as a nucleoside reverse transcriptase inhibitor, another nonnucleoside reverse transcriptase inhibitor or HIV protease inhibitor. When the active compound or its derivative or salt are administered in combination with another antiviral agent the activity may be increased over the parent compound. When the treatment is combination therapy, such administration may be concurrent or sequential with respect to that of the nucleoside derivatives. “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent.

It will be understood that references herein to treatment extend to prophylaxis as well as to the treatment of existing conditions, and that the treatment of animals includes the treatment of humans as well as other animals. Furthermore, treatment of a HIV infection, as used herein, also includes treatment or prophylaxis of a disease or a condition associated with or mediated by HIV infection, or the clinical symptoms thereof.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Example X

Heteropolymer HIV Reverse Transcriptase Assay: Inhibitor IC₅₀ Determination

HIV-1 RT assay was carried out in 96-well Millipore MultiScreen MADVNOB50 plates using purified recombinant enzyme and a poly(rA)/oligo(dT)₁₆ template-primer in a total volume of 50 μL. The assay constituents were 50 mM Tris/HCl, 50 mM NaCl, 1 mM EDTA, 6 mM MgCl₂, 5 μM dTTP, 0.15 μCi [³H] dTTP, 5 μg/ml poly (rA) pre annealed to 2.5 μg/ml oligo (dT)₁₆ and a range of inhibitor concentrations in a final concentration of 10% DMSO. Reactions were initiated by adding 4 nM HIV-1 RT and after incubation at 37° C. for 30 min, they were stopped by the addition of 50 μl ice cold 20% TCA and allowed to precipitate at 4° C. for 30 min. The precipitates were collected by applying vacuum to the plate and sequentially washing with 3×200 μl of 10% TCA and 2×200 μl 70% ethanol. Finally, the plates were dried and radioactivity counted in a Packard TopCounter after the addition of 25 μl scintillation fluid per well. IC₅₀'s were calculated by plotting % inhibition versus log₁₀ inhibitor concentrations. Representative IC₅₀ data is depicted in TABLE 2.

### Example Y

### Antiviral Assay Method

Anti-HIV-1 antiviral activity was assessed using an adaptation of the method of Pauwels et al. (Pauwels et al., J Virol Methods 1988 20:309-321). The method is based on the ability of compounds to protect HIV-1-infected T lymphoblastoid cells (MT4 cells) from cell-death mediated by the infection. The endpoint of the assay was calculated as the concentration of compound at which the cell viability of the culture was preserved by 50% (‘50% inhibitory concentration’, IC₅₀). The cell viability of a culture was determined by the uptake of soluble, yellow 3-[4,5-dimethylthiazol-2-yl]-2,5-diphenyltetrazolium bromide (MTT) and its reduction to a purple insoluble formazan salt. After solubilization, spectrophotometric methods were employed to measure the amount of formazan product.

MT4 cells were prepared to be in logarithmic-phase growth and a total of 2×10⁶ cells infected with the HXB2-strain of HIV-1 at a multiplicity of 0.0001 infectious units of virus per cell in a total volume of between 200-500 microliters. The cells were incubated with virus for one hour at 37° C. before removal of virus. The cells are then washed in 0.01 M phosphate buffered saline, pH 7.2 before being resuspensed in culture medium for incubation in culture with serial dilutions of test compound. The culture medium used was RPMI 1640 without phenol red, supplemented with penicillin, streptomycin, L-glutamine and 10% fetal calf serum (GM10).

Test compounds were prepared as 2 mM solutions in dimethyl sulfoxide (DMSO). Four replicate, serial 2-fold dilutions in GM10 were then prepared and 50 microliters amounts placed in 96-well plates over a final nanomolar concentration range of 625-1.22. Fifty microliters GM10 and 3.5×10⁴ infected cells were then added to each well. Control cultures containing no cells (blank), uninfected cells (100% viability; 4 replicates) and infected cells without compound (total virus-mediated cell death; 4 replicates) were also prepared. The cultures were then incubated at 37° C. in a humidified atmosphere of 5% CO₂ in air for 5 days.

A fresh solution of 5 mg/mL MTT was prepared in 0.01 M phosphate buffered saline, pH 7.2 and 20 microliters added to each culture. The cultures were further incubated as before for 2 hours. They were then mixed by pipetting up and down and 170 microliters of Triton X-100 in acidified isopropanol (10% v/v Triton X-100 in 1:250 mixture of concentrated HCl in isopropanol). When the formazan deposit was fully solubilized by further mixing, the absorbance (OD) of the cultures was measured at 540 nm and 690 nm wavelength (690 nm readings were used as blanks for artifacts between wells). The percent protection for each treated culture was then calculated from the equation:

$$\% Protection = \frac{\begin{matrix}( O D drug treated cultures ) - \\ ( O D untreated virus control cultures )\end{matrix}}{\begin{matrix}( O D uninfected cultures ) - \\ ( O D untreated virus control cultures )\end{matrix}} \times 100 \%$$

The IC₅₀ can be obtained from graph plots of percent protection versus log₁₀ drug concentration.

In both assays, compounds of Formulae I and II range in activity from an IC₅₀ of about 0.5 to about 10000 nM or 0.5 to about 5000 nM, with preferred compounds having a range of activity from about 0.5 to about 750 nM, more preferably about 0.5 to 300 nM, and most preferably about 0.5 to 50 nM.

TABLE 2

| | Polymerase | IC50 Antiviral |
|---|---|---|
| Compound# | Inhibition IC₅₀ (μM) | wt:fbs:10% (μM) |
| II-3 | 0.0056 | 0.0033 |
| II-6 | 0.0073 | 0.0024 |
| II-18 | 0.0004 | 0.0042 |
| I-19 | 0.0063 | 0.0069 |
| I-20 | 0.0015 | 0.0044 |

### Example Z

### Pharmaceutical Compositions

Pharmaceutical compositions of the subject Compounds for administration via several routes were prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. | |
|---|---|---|
| Active ingredient | 0.25 | g |
| Sodium Chloride | qs to make isotonic | |
| Water for injection to | 100 | ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation (E)

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Topical Formulation (F)

| Ingredients | grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy | 0.01 |
| anisole) | |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed is:

1. A compound of Formula I 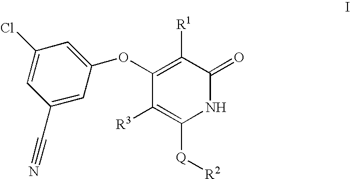
wherein:
R¹ is halogen, lower alkyl, lower alkenyl, or amino;
Q is Q¹ or Q²;
Q¹ is lower alkylene;
Q² is Q-Q³;
Q³ is —C(═O)—;
R² is phenyl, heteroaryl, or heterocycloalkyl, optionally substituted with one or more R²;
R^{2′} is lower alkyl or halogen; and
R³ is H, halogen, or lower alkyl.

2. The compound of claim 1, wherein R¹ is halogen.

3. The compound of claim 2, wherein Q is ethylene.

4. The compound of claim 3, wherein R² is phenyl.

5. The compound of claim 3, wherein R² is pyridyl.

6. The compound of claim 1, wherein Formula I is selected from the group consisting of:
3-Chloro-5-{6-[2-(3,4-dihydro-1H-isoquinolin-2-yl)-2-oxo-ethyl]-3-dimethylamino-2-oxo-1,2-dihydro-pyridin-4-yloxy}-benzonitrile;
3-[6-(2-Benzooxazol-2-yl-ethyl)-3-dimethylamino-5-methyl-2-oxo-1,2-dihydro-pyridin-4-yloxy]-5-chloro-benzonitrile;
3-(3-Bromo-2-oxo-6-phenethyl-1,2-dihydro-pyridin-4-yloxy)-5-chloro-benzonitrile;
3-Chloro-5-(3-chloro-2-oxo-6-phenethyl-1,2-dihydro-pyridin-4-yloxy)-benzonitrile;
3-{3-Bromo-6-[2-(3,4-dihydro-1H-isoquinolin-2-yl)-2-oxo-ethyl]-5-fluoro-2-oxo-1,2-dihydro-pyridin-4-yloxy}-5-chloro-benzonitrile;
3-(3-Bromo-5-fluoro-2-oxo-6-phenethyl-1,2-dihydro-pyridin-4-yloxy)-5-chloro-benzonitrile;
3-Chloro-5-(3-chloro-5-fluoro-2-oxo-6-phenethyl-1,2-dihydro-pyridin-4-yloxy)-benzonitrile;
3-Chloro-5-{3-chloro-6-[2-(2-methyl-pyridin-4-yl)-ethyl]-2-oxo-1,2-dihydro-pyridin-4-yloxy}-benzonitrile;
3-Chloro-5-{6-[2-(3-chloro-phenyl)-ethyl]-5-fluoro-3-iodo-2-oxo-1,2-dihydro-pyridin-4-yloxy}-benzonitrile;
3-Chloro-5-[3-chloro-2-oxo-6-(2-m-tolyl-ethyl)-1,2-dihydro-pyridin-4-yloxy]-benzonitrile;
3-Chloro-5-[3-chloro-2-oxo-6-(2-pyridin-4-yl-ethyl)-1,2-dihydro-pyridin-4-yloxy]-benzonitrile;
3-{3-Bromo-6-[2-(3-chloro-phenyl)-ethyl]-5-fluoro-2-oxo-1,2-dihydro-pyridin-4-yloxy}-5-chloro-benzonitrile;
3-Chloro-5-[3-chloro-2-oxo-6-(2-pyridin-3-yl-ethyl)-1,2-dihydro-pyridin-4-yloxy]-benzonitrile;
3-Chloro-5-[3-chloro-2-oxo-6-(2-pyridin-2-yl-ethyl)-1,2-dihydro-pyridin-4-yloxy]-benzonitrile;
3-Chloro-5-{3-chloro-6-[2-(3-chloro-phenyl)-ethyl]-2-oxo-1,2-dihydro-pyridin-4-yloxy}-benzonitrile;
3-Chloro-5-{3-chloro-6-[2-(3,4-dihydro-1H-isoquinolin-2-yl)-2-oxo-ethyl]-2-oxo-1,2-dihydro-pyridin-4-yloxy}-benzonitrile;
3-{3-Bromo-5-fluoro-6-[2-(3-fluoro-phenyl)-ethyl]-2-oxo-1,2-dihydro-pyridin-4-yloxy}-5-chloro-benzonitrile;
3-Chloro-5-{3-chloro-5-fluoro-6-[2-(3-fluoro-phenyl)-ethyl]-2-oxo-1,2-dihydro-pyridin-4-yloxy}-benzonitrile;
3-[6-(2-Benzooxazol-2-yl-ethyl)-3-chloro-2-oxo-1,2-dihydro-pyridin-4-yloxy]-5-chloro-benzonitrile;
3-[3-Bromo-5-fluoro-2-oxo-6-(2-pyridin-4-yl-ethyl)-1,2-dihydro-pyridin-4-yloxy]-5-chloro-benzonitrile;
3-Chloro-5-[3-chloro-5-fluoro-2-oxo-6-(2-pyridin-4-yl-ethyl)-1,2-dihydro-pyridin-4-yloxy]-benzonitrile; and
3-f{3-Bromo-6-[2-(3-chloro-phenyl)-ethyl]-5-fluoro-2-oxo-1,2-dihydro-pyridin-4-yloxy}-5-chloro-benzonitrile.

7. A compound of Formula II 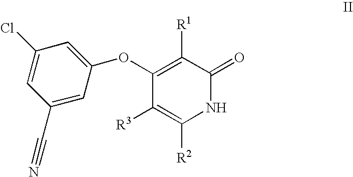
wherein:
R¹ is halogen, lower alkyl, lower alkenyl, or amino;
R² is H or lower alkyl;
R³ is —R⁴ or —R—R⁶;
R⁴ is lower alkyl;
R⁵ is —(CH₂)ₘ—, —(CH₂)ₘO— or —(CH₂)ₘS—;
m is 1, 2, or 3;
R⁶ is phenyl, phenyl lower alkylenyl, heteroaryl, or heteroaryl lower alkylenyl, optionally substituted with one or more R⁶; and
R^{6′} is lower alkyl, halogen or lower alkoxy.

8. The compound of claim 7, wherein R¹ is halogen.

9. The compound of claim 8, wherein R³ is —R⁵—R⁶, R⁵ is —(CH₂)ₘO—, and m is 2.

10. The compound of claim 8, wherein R³ is —R⁵—R⁶, R⁵ is —(CH₂)ₘ—, and m is 2.

11. The compound of claim 8, wherein R³ is —R⁵—R⁶, R⁵ is —(CH₂)ₘ—, and m is 3.

12. The compound of claim 8, wherein R³ is —R⁵—R⁶, R⁵ is —(CH₂)ₘS—, and m is 1.

13. The compound of claim 7, wherein Formula II is selected from the group consisting of:
3-Chloro-5-[3-dimethylamino-2-oxo-5-(3-phenyl-propyl)-1,2-dihydro-pyridin-4-yloxy]-benzonitrile;
3-(3-Bromo-5-ethyl-2-oxo-1,2-dihydro-pyridin-4-yloxy)-5-chloro-benzonitrile;
3-Chloro-5-(5-ethyl-2-oxo-3-vinyl-1,2-dihydro-pyridin-4-yloxy)-benzonitrile;
3-Chloro-5-(3-chloro-5-ethyl-2-oxo-1,2-dihydro-pyridin-4-yloxy)-benzonitrile;
3-(3-Bromo-2-oxo-5-propyl-1,2-dihydro-pyridin-4-yloxy)-5-chloro-benzonitrile;
3-(3-Bromo-5-ethyl-6-methyl-2-oxo-1,2-dihydro-pyridin-4-yloxy)-5-chloro-benzonitrile;
3-[3-Bromo-2-oxo-5-(2-phenoxy-ethyl)-1,2-dihydro-pyridin-4-yloxy]-5-chloro-benzonitrile;
3-(5-Benzyl-3-bromo-2-oxo-1,2-dihydro-pyridin-4-yloxy)-5-chloro-benzonitrile;
3-{3-Bromo-2-oxo-5-[2-(pyridin-3-yloxy)-ethyl]-1,2-dihydro-pyridin-4-yloxy}-5-chloro-benzonitrile;
3-(5-Benzyl-3-bromo-6-methyl-2-oxo-1,2-dihydro-pyridin-4-yloxy)-5-chloro-benzonitrile;
3-[3-Bromo-6-methyl-2-oxo-5-(3-phenyl-propyl)-1,2-dihydro-pyridin-4-yloxy]-5-chloro-benzonitrile;
3-{3-Bromo-2-oxo-5-[2-(pyridin-4-yloxy)-ethyl]-1,2-dihydro-pyridin-4-yloxy}-5-chloro-benzonitrile;
3-Chloro-5-[3-chloro-6-methyl-2-oxo-5-(3-pyridin-4-yl-propyl)-1,2-dihydro-pyridin-4-yloxy]-benzonitrile;
3-Chloro-5-[3-chloro-6-methyl-2-oxo-5-(3-pyridin-2-yl-propyl)-1,2-dihydro-pyridin-4-yloxy]-benzonitrile;
3-[3-Bromo-2-oxo-5-(pyridin-4-ylmethoxymethyl)-1,2-dihydro-pyridin-4-yloxy]-5-chloro-benzonitrile;
3-Chloro-5-[3-chloro-6-methyl-2-oxo-5-(2-phenoxy-ethyl)-1,2-dihydro-pyridin-4-yloxy]-benzonitrile;
3-Chloro-5-[3-chloro-6-methyl-2-oxo-5-(3-pyridin-3-yl-propyl)-1,2-dihydro-pyridin-4-yloxy]-benzonitrile;
3-(5-Benzylsulfanylmethyl-3-bromo-2-oxo-1,2-dihydro-pyridin-4-yloxy)-5-chloro-benzonitrile;
3-Chloro-5-[3-chloro-6-methyl-2-oxo-5-(3-pyrimidin-4-yl-propyl)-1,2-dihydro-pyridin-4-yloxy]-benzonitrile; and
3-Chloro-5-[3-chloro-6-methyl-2-oxo-5-(3-pyridazin-3-yl-propyl)-1,2-dihydro-pyridin-4-yloxy]-benzonitrile.

14. A method of treating a disease associated with HIV comprising administering to a patient in need thereof, a therapeutically effective amount of the compound of claim 1.

15. A method of treating a disease associated with HIV comprising administering to a patient in need thereof, a therapeutically effective amount of the compound of claim 7.

16. A method for preparing a compound of Formula Ia, 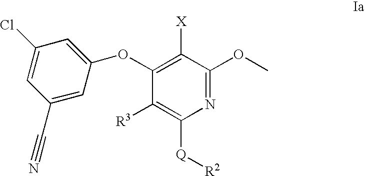
wherein:
X is halide;
Q is Q¹ or Q²;
Q¹ is lower alkylene;
Q² is Q¹-Q³;
Q³ is —C(═O)—;
R² is phenyl, heteroaryl, or heterocycloalkyl, optionally substituted with one or more R^{2′};
R² is lower alkyl or halogen; and
R³ is H, halogen, or lower alkyl;
comprising the steps of:
a) treating a solution of cupric halide and lithium halide with tert-Butyl nitrite;
b) treating the product of step a) with a compound of Formula Ib; 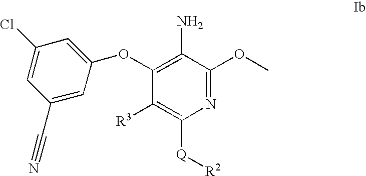
c) treating the product of step b) with an aqueous hydrohalic acid solution.

17. A method for preparing a compound of Formula Ia, 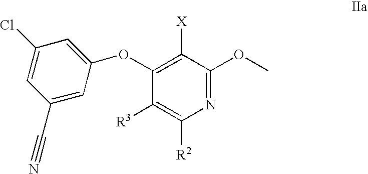
wherein:
R² is H or lower alkyl;
R³ is —R⁴ or —R⁵—R⁶;
R⁴ is lower alkyl;
R⁵ is —(CH₂)ₘ—, —(CH₂)ₘO— or —(CH₂)ₘS—;
m is 1, 2, or 3;
R⁶ is phenyl, phenyl lower alkylenyl, heteroaryl, or heteroaryl lower alkylenyl, optionally substituted with one or more R^{6′}; and
R^{6′} is lower alkyl, halogen or lower alkoxy;
comprising the steps of:
a) treating a solution of cupric halide and lithium halide with tert-Butyl nitrite;
b) treating the product of step a) with a compound of Formula IIb; 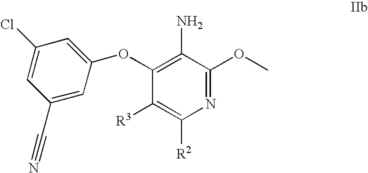
c) adding an aqueous hydrohalic acid solution the product of step b).
